(19) United States
(12) Patent Application Publication
(10) Pub. No.: US 2019/0227087 A1
(43) Pub. Date: Jul. 25, 2019

(54) CLOUD-BASED DIGITAL ROCK ANALYSIS AND DATABASE SERVICES

(71) Applicant: SCHLUMBERGER TECHNOLOGY CORPORATION, Sugar land, TX (US)

(72) Inventors: Ashok K. Belani, Sugar Land, TX (US); Sergey Sergeevich Safonov, Moscow (RU); Oleg Yurievich Dinariev, Moscow (RU); Denis Vladimirovich Klemin, Houston, TX (US); Leonid Dovgilovich, Moscow (RU); Vasily Baydin, Chelyabinsk (RU)

(21) Appl. No.: 15/772,766

(22) PCT Filed: Nov. 1, 2016
(86) PCT No.: PCT/US2016/059958

§ 371 Date: May 1, 2018

Related U.S. Application Data
(60) Provisional application No. 62/249,755, filed on Nov. 2, 2015.

(51) Int. Cl. G01N 35/00; G01N 33/24

(52) U.S. Cl. CPC G01N 35/00871; G01N 2035/00881; G01N 33/24

(57) ABSTRACT

A method includes receiving, via an network interface of a cloud-based infrastructure, a request for analysis of rock material properties based at least in part on a digital, image-based model of the rock material; responsive to the request, executing the analysis via provisioning of one or more resources of the cloud-based infrastructure to generate analysis results; and transmitting information based at least in part on the analysis results.

### BACKGROUND

Rock can be defined as an aggregate of minerals or organic matter (e.g., consider coal) or volcanic glass (e.g., consider obsidian). Rock can include a single mineral, such as rock salt (e.g., halite) and certain limestones (e.g., calcite) or more than one mineral such as, for example, granite (e.g., quartz, feldspar, mica and other minerals). Types of rock can include, for example, sedimentary, igneous and metamorphic. Sedimentary rocks like sandstone and limestone tend to form at the Earth's surface through deposition of sediments derived from weathered rocks, biogenic activity or precipitation from solution. Igneous rocks tend to originate deeper within the Earth, where the temperature is high enough to melt rocks, to form magma that can crystallize within the Earth or at the surface by volcanic activity. Metamorphic rocks tend to form from other preexisting rocks during episodes of deformation of the Earth at temperatures and pressures high enough to alter minerals but inadequate to melt them. Such changes can occur by the activity of fluids in the Earth and movement of igneous bodies or regional tectonic activity. Rock can be recycled from one type to another by changes in the Earth.

Rock may form a reservoir that can include fluid or fluids such as, for example, fluid or fluids that include water, hydrocarbons, etc. A reservoir can be a subsurface body of rock having sufficient porosity and permeability to store and transmit fluid(s). Sedimentary rocks can be reservoir rocks as they tend to have more porosity than various igneous rocks and metamorphic rocks. Sedimentary rocks tend to form under temperature conditions at which hydrocarbons can be preserved (e.g., as in a petroleum system).

Exploration can be a phase in petroleum operations that includes, for example, generation of a prospect or play or both, and drilling of one or more exploration wells. One or more phases may follows such as, for example, appraisal, development and production phases may follow successful exploration.

Core sampling and analysis may occur during one or more phases of operations. Core analysis can include laboratory study of a sample of a geologic formation (e.g., reservoir rock or other rock), taken during or after drilling a well. Economic and efficient oil and gas production can depend on understanding properties of reservoir rock such as, for example, porosity, permeability, and wettability. Various types of log and/or core analysis techniques may be utilized to measure one or more of such properties. Core analysis for shale reservoirs can elucidate vertical and lateral heterogeneity of the rocks. Core analysis can include evaluation of rock properties and anisotropy; organic matter content, maturity, and type; fluid content; fluid sensitivity; and geomechanical properties. Such examples of information may be used, for example, to calibrate log and/or seismic measurements and to help in well and completion design, well placement, and other aspects of reservoir production.

### SUMMARY

A method can include receiving, via an network interface of a cloud-based infrastructure, a request for analysis of rock material properties based at least in part on a digital, image-based model of the rock material; responsive to the request, executing the analysis via provisioning of one or more resources of the cloud-based infrastructure to generate analysis results; and transmitting information based at least in part on the analysis results. A system can include servers where each of the servers includes at least one processor, memory accessible by the at least one processor and processor-executable instructions stored in the memory to analyze rock material properties based on a digital, image-based model of the rock material to generate analysis results; a network interconnect where the servers are operatively coupled to the network interconnect; provisioning circuitry that provisions the servers responsive to receipt of a request to analyze the rock material properties; and transmission circuitry that transmits information based at least in part on the analysis results. One or more computer-readable storage media can include computer-executable instructions to instruct a computing system to: receive, via an network interface of a cloud-based infrastructure, a request for analysis of rock material properties based at least in part on a digital, image-based model of the rock material; responsive to the request, execute the analysis via provisioning of one or more resources of the cloud-based infrastructure to generate analysis results; and transmit information based at least in part on the analysis results. Various other apparatuses, systems, methods, etc., are also disclosed.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

Features and advantages of the described implementations can be more readily understood by reference to the following description taken in conjunction with the accompanying drawings.

FIG. 1 illustrates an example of a system;

FIG. 2 illustrates various examples of components of the system of FIG. 1;

FIG. 3 illustrates an example of a system;

FIG. 4 illustrates an example of a method and an example of a system;

FIG. 5 illustrates an example of a simulation modeling tool;

FIG. 6 illustrates an example of an output component;

FIG. 7 illustrates examples of components of a cloud-based digital rock system;

FIG. 8 illustrates an example of a geologic environment, an example of a plot and an example of a chart;

FIG. 9 illustrates examples of equipment in a geologic environment, an example of a system and an example of a toolstring;

FIG. 10 illustrates an example of a graphical user interface;

FIG. 11 illustrates an example of a method;

FIG. 12 illustrates an example of a method; and

FIG. 13 illustrates example components of a system and a networked system.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims.

A cloud-based digital rock simulation and database system can include services that provide for digital information handling, storage and simulation associated with various digital rock workflows as may be used, for example, for petrophysical and multiphase flow property evaluation, reservoir characterization, hydrocarbon production analysis, etc.

In a cloud-based environment, such a system can be accessible by a plurality of different enterprises and users. A cloud-based architecture can be adaptable in real-time within a hardware infrastructure to receive a request for digital data processing, execute a massive pore-scale simulation in response to the request (e.g., from a particular one of a plurality of enterprises), store and handle information on digital rock and fluid data, simulation results and executed scenario(s), and report processed data that can include time-dependent multi-parameter three-dimensional graphical output related to one or more performed simulations and stored information.

As an example, a cloud-based system can include various features for physical and digital rock and fluid analyses, which may aid in creating a reservoir model for simulation of flow performance under multiple production scenarios. Such a system may utilize physical laboratory measurements to refine reservoir simulation, for example, to enhance determinations as to relative permeability, capillary pressure, net present value, and other parameters associated with reservoir engineering. As an example, fluid can include liquid and/or gas.

As an example, a system may include one or more features of the COREFLOW™ framework (Schlumberger Limited, Houston, Tex.). Such a framework can include instructions that are executable to perform digital simulations. As an example, physical laboratory measurements and/or physical measurements can be utilized to refine digital simulations. As an example, analyses of fluid properties can be performed to create digital fluid models for flow simulation.

While the aforementioned framework refers to “cores”, it can also analyze materials such as proppant as well as interactions between proppant, chemicals, fluids, etc. Proppant can be sized particles mixed with fracturing fluid to hold fractures open after a hydraulic fracturing treatment. Proppant may include naturally occurring sand grains, man-made or specially engineered particles such as, for example, resin-coated sand or high-strength ceramic materials like sintered bauxite. Proppant materials can be sorted for size and sphericity to provide an efficient conduit for production of fluid from a reservoir to a wellbore.

Cloud computing services can be provided via information technology (IT) instruments and technologies that are made available to users in an on-demand manner via the Internet. Cloud computing can allow companies to consume compute resources as a utility rather than having to build and maintain computing infrastructures in-house. Cloud services can provide relatively easy, flexible and scalable access to computing applications, resources and services, and may be managed by a cloud services provider.

An example of a cloud services provider is Amazon Web Services (AWS™, Amazon.com, Seattle, Wash.), which offers a suite of cloud-computing services that make up an on-demand computing platform. AWS™ services operate from over a dozen geographical regions across the world. AWS™ services include Amazon Elastic Compute Cloud, also known as “EC2”, and Amazon Simple Storage Service, also known as “S3”. AWS™ services include compute, storage, networking, database, analytics, application services, deployment, management, mobile, developer tools and tools for the Internet of things. AWS™ services can provide large computing capacity as an alternative to a user having to build a physical server farm.

As an example, a cloud computing platform can be utilized to implement a cloud-based system. For example, consider the AZURE™ platform (Microsoft Corporation, Redmond, Wash.), which is a cloud computing platform and infrastructure for building, deploying, and managing applications and services through a global network of data centers.

As an example, a cloud-based infrastructure can include features for Web apps, app services, virtual machines, storage, one or more SQL database, etc. As an example, a cloud-based infrastructure can include a cloud computing platform that can be accessible and responsive to information received via one or more interfaces (e.g., network interfaces), which may operate according to one or more application programming interfaces (APIs). As an example, information can be output to a client device such as, for example, a dashboard, a chart, images, video, etc.

As an example, a cloud-based infrastructure can provide for creation of virtual machines for on-premises servers and/or scale-up to help balance resources and increase available, timing, etc., of one or more applications. As an example, virtual machines can integrate cloud-based infrastructure capacity into a datacenter or datacenters (e.g., for global load balancing, etc.) as desired and/or may provide access to on-demand HPC capabilities in a cloud-based infrastructure, optionally in a scalable manner where a request may be “sized” and resources provisioned to provide analysis results within a desired amount of time. For example, where field operations are to utilize analysis results for purposes of decision making, control, etc., resources may be provisioned that can provide analysis results (e.g., simulation results, etc.) in a desired time frame for purposes of decision making in the field. In such an example, feedback may be provided to the resources based on field data such that convergence is sought between field data and analysis results (e.g., simulation results). Such an approach can allow for enhanced decision making in the field and can allow for learning as to modeling, simulation, etc., of a “smart” system implemented at least in part via a cloud-based infrastructure. A cloud-based infrastructure can include one or more learning algorithms (e.g., neural network, etc.) that aim to increase efficiency and/or accuracy of one or more analyses as one or more digital rock analysis and/or database services are utilized (e.g., via client devices in one or more laboratories, one or more fields, one or more mobile pieces of equipment, etc.). A cloud-based infrastructure can include de-provisioning, for example, once a request has been satisfied (e.g., a session halted or terminated).

A cloud computing platform can offer, for example, virtual machines, infrastructure as a service (laaS) that provide for launch of virtual machines and/or preconfigured machine images, App services, a platform as a service (PaaS) environment (e.g., to publish and/or manage Web sites), Websites, high density hosting of websites (e.g., optionally using one or more of ASP.NET, PHP, Node.js, Python, etc.), etc. As an example, a cloud-based system may utilize Websites in PHP, ASP.NET, Node.js, Python, or one or more other languages. As an example, a cloud computing platform may offer WebJobs as applications that can be deployed to a Web App to implement background processing. Such an approach may be invoked on a schedule, on-demand and/or run continuously. As an example, a cloud computing platform may offer blob (data storage/structure), table and queue services, which may be utilized to communicate between Web Apps and WebJobs and, for example, to provide state information.

A cloud computing platform can provide one or more of SaaS, PaaS and laaS services and, for example, supports different programming languages, tools and frameworks.

Cloud computing can allow users to benefit from various computing technologies, optionally without deep knowledge about or expertise with each one of them. Cloud computing can reduce, manage and/or control costs. Implementation in a cloud environment can help a service provider to focus on business instead of being impeded by IT obstacles.

As mentioned, cloud services can dynamically scale, for example, to meet demands of users. Provisioning may be automated in a cloud environment where a cloud infrastructure provider supplies hardware and software.

Could computing can be defined in part via the following three application categories: infrastructure as a service (IaaS), platform as a service (PaaS) and software as service (SaaS). As to SaaS, digital rock simulation services and associated digital rock database services can be exposed via the Internet, which can allow customers to use simulation technology as a service in the cloud. Such an approach can mitigate customer costs and allow for on-demand simulation to enhance their productivity as to one or more phases of operations associated with one or more reservoirs.

As an example, the aforementioned COREFLOW™ framework can be hosted in a cloud environment, for example, via a cloud computing platform. In such an example, a cloud-based system can provide digital rock simulation services and associated database services. As an example, one or more reservoir engineers can access one or more of such services via a Web portal or portals to help address current challenges in the petrophysics and reservoir engineering. As an example, a core analyst or core analysts can access one or more of such services to help understand and realistically model pore geometries and fluid behaviors at pores scales in a timesaving manner.

As an example, a cloud-based system can allow for collaboration and teamwork in performing one or more workflows germane to field operations. As an example, a user may commence a workflow via interaction between a computing device and a cloud-based system where results therefrom are distributed to one or more other users (e.g., via appropriate computing equipment). Such an approach may allow for hand-offs where progress of a workflow or workflows may be monitored and/or managed, optionally in real-time, in a just-in-time (JIT) basis, etc., because interactions, computing and data handling occur in a common cloud computing platform.

As an example, a cloud computing system can include components for implementing digital rock analysis that integrates physical and digital core techniques, for example, using one or more common rock samples for both types of analysis. Using such a service, oil and gas operators can shorten traditional cycle times, understand better one or more reservoirs prior to making one or more field decisions, and maximize short-term production and long-term recover from oil and gas assets worldwide.

The aforementioned COREFLOW™ framework is an example of a framework that offers digital rock services for reservoir characterization and hydrocarbon production analysis. A framework can provide an integrated solution that creates a pore-scale 3D reservoir model to rapidly simulate flow performance under multiple production scenarios and deliver an actionable digital fluid model for use in making various decisions (e.g., as to drilling, production, stimulation, etc.). As an example, physical measurements can be utilized to refine a digital rock model, while digital flow scenarios can guide subsequent lab tests in an iterative manner, rather than in a sequential series of steps.

As an example, complex multiphase pore-scale flow simulations can be carried out using direct hydrodynamic (DHD) simulation. As an example, a DHD simulator can be based on a density functional (DF) method applied for multiphase compositional hydrodynamics. Such a simulator can combines continuum fluid mechanics and thermodynamic principles by considering mass, momentum and energy balance together with a diffuse interface description. The diffuse interface approach is a physically consistent and efficient for modeling evolution of fluid-fluid interfaces in multiphase flow. A DHD simulator can combines concepts from physical chemistry, statistical physics and physics of solids with hydrodynamics and takes into account interfacial surface tension, interfacial tension at contact with solid surfaces (wettability), moving contact lines and dynamic changes of topology of interfaces.

A DHD simulator may be implemented for modelling hydrodynamics such as, for example, complex compositional fluids with phase transitions (gas-liquid, liquid-liquid, liquid-solid); flow in complex geometries of boundary surfaces; wettability and adsorption; surfactants, solvents, polymers; complex fluid rheology and presence of mobile solid phase; and thermal effects. Fluid phase behavior, which is traditionally characterized by an equation of state (EoS), can be handled as a thermodynamic fluid model (e.g., specified by Helmholtz free energy functions).

High performance computing with a massively parallel GPU realization of DHD code together with enhanced algorithms of cross-machine and cross-GPU communications interleaved with computations, can allow for modelling several tens of billions (˜10¹⁰) of cells on a medium-sized GPU cluster. Characteristic computational times for complex multiphase flows in representative sub-volumes of digitized rock samples may be of the order of 24 hours while simpler geometries may be of the order of minutes. As an example, a DHD simulator may be implemented in a cloud environment using a cloud computing platform. As an example, a DHD simulator may be implemented in a distributed manner using various computing resources available in a cloud environment.

A DHD simulator can be implemented to understand better flow and parameters related to flow and operations that can be utilized to adjust flow (e.g., enhance flow, prolong flow, etc.). A direct hydrodynamics pore-scale flow simulation can simulate flow in porous media that can lead to improved recovery of hydrocarbons in the field. Such an approach can be dynamic modeling and provide a more comprehensive reservoir understanding based on fundamental physics. Simulation results can allow for reservoir characterization to facilitate, for example, reserves estimation, and offers a level of detail in modeling that can be used to improve production scenario planning decisions for optimized hydrocarbon recovery.

A DHD direct hydrodynamic pore-scale flow simulation approach can combine digital rock models, digital fluid models, 3D wettability distribution, and setup of boundary conditions to simulate fluid flow through porous media. DHD simulation can generate data on capillary pressure, absolute and relative permeability, recovery efficiency, and flow heterogeneity. DHD simulation can be used for modeling geomechanical property response of a digital rock model to loading, petrophysical property processes modeling and/or one or more other types of property analysis (e.g., thermal, NMR, electric and acoustic properties, etc.). As an example, a workflow can include combining simulations with laboratory measurements of properties to yield better reservoir answers faster than with digital or physical measurements alone.

DHD simulations can represent real pore geometries, real fluid properties, and real rock-fluid/fluid-fluid behaviors, without oversimplifying assumptions as to these components. A cloud-based system can allow for shortening of cycle times, better understanding of increasingly complex reservoirs (e.g., before making costly field decisions), and maximizing both short-term production and long-term recover from oil and gas assets worldwide.

A cloud-based system for digital rock simulation and associated data handling services can address increasing complexity of reservoir formation, fluid behavior and recovery methods. Such a system can provide digital rock simulations in a practically feasible spatial domain and time scale. A cloud-based system can be accessible via the Internet according to appropriate entity accounts (e.g., entity log-in and access credentials).

As an example, a cloud-based system can include digital rock models for generating simulation results via one or more simulators (e.g., which may be instances of simulators in a cloud environment). In such an example, simulation results can include a substantial amount of numerical information and associated data that can be properly and securely accumulated and stored, for example, to provide for analysis, archiving and/or retrieval. Digital image data of rock material can include a set of digital core images, results of mineral mapping of one or more rock samples, results of representative elementary volume analysis of one or more rock samples, results of microporosity analysis of one or more rock samples, results of wettability mapping of one or more rock samples, results of microstructural and heterogeneity analysis by NMR/MRI of one or more rock samples, results of geomechanical analysis of one or more rock samples, etc. As an example, digital core images may be acquired via X-ray microtomography and/or by 3D NMR imaging and/or be reconstructed using petrographic thin-section analysis data and/or SEM data, optionally with application of one or more image analysis techniques (e.g., for binarization of grayscale or color 2D slices, etc.).

A cloud-based system can include a process architecture and corresponding infrastructure for digital rock and fluid data handling, storing and efficient numerical simulations supported by cloud-based high performance computing technology with a capability for remote access and control via the Internet.

A cloud-based system can be accessible, interactively, via networked client devices, such as workstation, desktop computers, laptops, tablets and mobile phones. Customer access can also be applied for remote visualization of 2D and 3D initial, processed and simulated images where image rendering may be performed, at least in part, by a cloud server with dedicated software and hardware. As an example, one or more cloud-based servers can receive one or more viewpoint requests from a device of a customer and, in response, transmit one or more corresponding 2D or 3D rendered images back to the device of the customer.

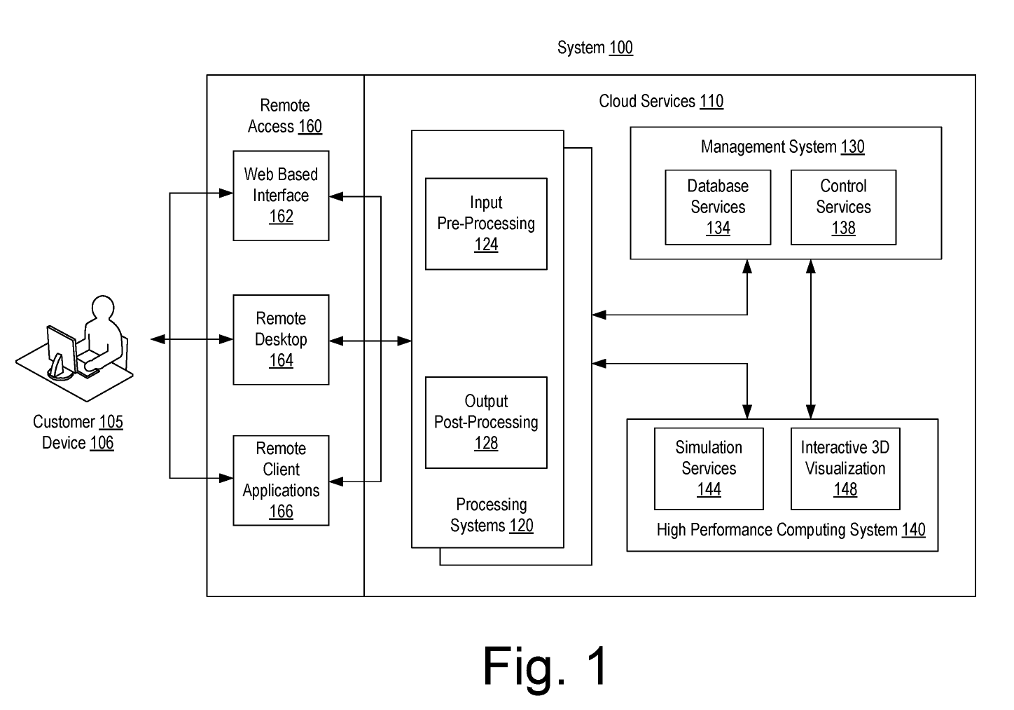

FIG. 1 shows an example of a system 100 that includes a customer 105 with an associated device 106, cloud services components 110 and remote access components 160. The cloud services components 110 include processing systems components 120, management system components 130 and high performance computing system (HPCS) components 140 while the remote access components 160 include a web-based interface component 162, a remote desktop component 164 and one or more remote client application components 166.

As shown in the example of FIG. 1, the device 106 as operated by the customer 105 can access one or more services of the cloud services components 110 via one or more of the remote access components 160. In such an example, the device 106 can include one or more network interfaces (e.g., WiFi, cellular, cable, etc.) that can operatively couple the device 106 to the Internet where the remote access components 160 are operatively coupled to the Internet, for example, via one or more network interfaces.

In the example of FIG. 1, the processing systems components 120 include an input pre-processing component 124 and an output post-processing component 128. As mentioned, a flow simulator can generate a substantial amount of data, particularly for time-dependent flows. As an example, the customer 105 may access data via the device 105 where the data can be in one or more formats. In such an example, the pre-processing component 124 can provide for formatting, parsing, processing, etc. data as input and, for example, the post-processing component 128 can provide for formatting, consolidating, processing, etc. data as output.

In the example of FIG. 1, the management system 130 includes a database services component 134 and a control services component 138 and the high performance computing system 140 includes a simulation services component 144 and an interactive 3D visualization component 148. The simulation services component 144 provides for flow simulation services such as, for example, image-based model building where a digital model can be built from multidimensional images of one or more pieces of rock (e.g., one or more core samples, etc.). As an example, a digital model can be built by rendering a set of laboratory measurement data. In one or more embodiments, laboratory data can be acquired via employing a high resolution technology (e.g., focus ion beam scanning electron microscope (FIB-SEM), SEM 2D scans, etc.) to scan a subsurface formation sample (e.g., a core sample or rock sample) and reproduce the spatial distribution of rock grains, pores and/or solid organics within the subsurface formation sample. In one or more embodiments, a digital model built by rendering may utilize a lower resolution technology (e.g., X-ray micro-tomography, CT scanning, etc.) to scan a subsurface formation sample and, for example, obtain heterogeneity information at a larger scale. The interactive 3D visualization component 148 can provide for generating visualization information (e.g., vector information, image information, etc.) for a model, models, simulation results and/or results based at least in part on simulation results. As mentioned, a viewpoint may be received or, for example, a path sequence whereby the interactive 3D visualization component 148 responds by generating visualization information that can be rendered to a display of the device 106 of the customer 105. As an example, a video file may be generated and/or streamed that shows flow and/or other behavior with respect to time or a change in a parameter, etc. (e.g., stress, porosity, viscosity, phase, etc.).

A workflow can include receiving a request for a simulation of fluid flow in rock via a network interface associated with a cloud-based system, processing the request, accessing information, performing the simulation of fluid flow in rock and transmitting results of the simulation via a network interface associated with the cloud-based system. In such an example, information may be received during the workflow and/or information may be transmitted during the workflow such that a user may monitor progress of the workflow, interact with the cloud-based system, etc.

As an example, a cloud-based digital rock simulation and database services system can include components for remote access which will help a customer to connect and interact with the system from various interfaces including web-based interfaces, remote desktops and remote client applications.

As an example, a cloud-based digital rock simulation and database services system can include components for a processing system that can form a request for digital rock simulation and/or related data storing/withdraw as supported by input data, which may be specified for a corresponding simulation. Such a processing system can generate output information, for example, in a customer's defined manner for remote utilization and assessment.

As an example, a cloud-based digital rock simulation and database services system can include components for a management system that can control and manage data storage, withdraw and data flow between a processing system, one or more databases, one or more simulation servers and one or more visualization servers.

As an example, a cloud-based digital rock simulation and database services system can include components for a high performance computing system or system that can run digital rock simulations (e.g., in an optimized and efficient manner).

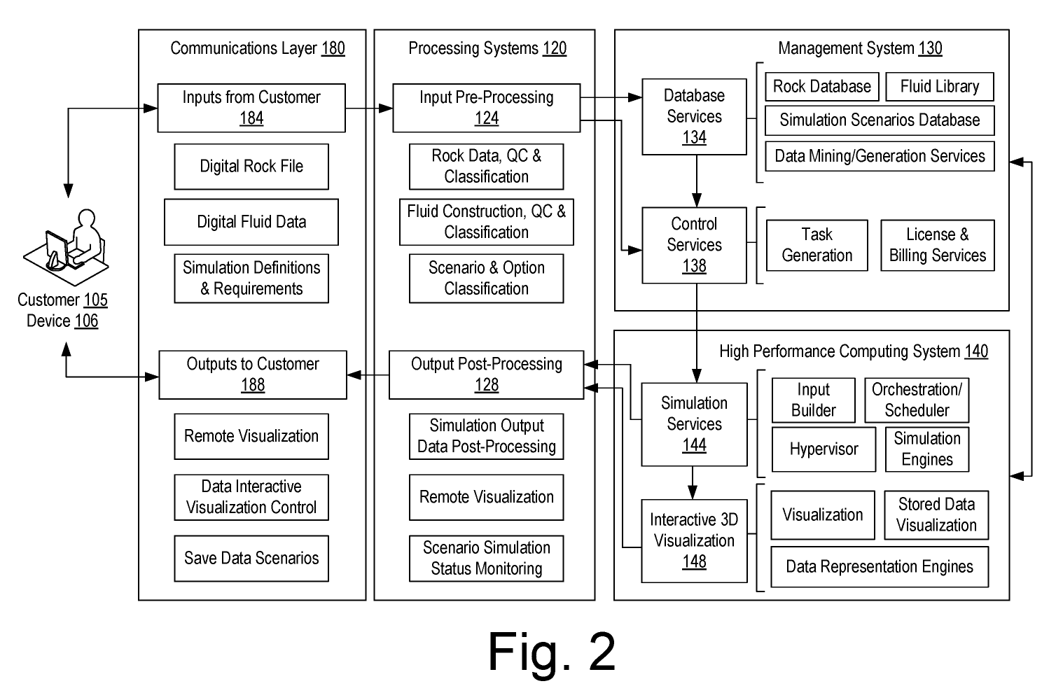

FIG. 2 shows an example of the cloud services components 110 of FIG. 1 along with examples of inputs and outputs in a communications layer 180 for communications from and to a customer 105 with an associated device 106. As shown, the communications layer 180 includes an input from customer component 184 that can handle digital rock files, digital fluid data and simulation definitions and requirements as well as an output to customer component 188 that can handle remote visualization, data interactive visualization control and saving of one or more data scenarios.

In the example of FIG. 2, the input pre-processing component 124 can include features to handle rock data, quality control (QC) and classification; fluid construction, quality control (QC) and classification; and scenario and option classification. For example, the input pre-processing component 124 can receive information from the input from customer component 184 and process such information for purposes of input to one or more components of the management system components 130 (e.g., the database services component 134 and/or the control services component 138).

In the example of FIG. 2, the output post-processing component 128 can include features to handle simulation output data post-processing, remote visualization and scenario simulation status monitoring. As shown, the output post-processing component 128 can receiving information from one or more components of the high performance computing system components 140 (e.g., the simulation services component 144 and/or the interactive 3D visualization component 148).

In the example of FIG. 2, the database services component 134 is shown as including, for example, rock data features, fluid library features, simulation scenario database features and data mining and/or data generation services features while the control services component 138 is shown as including, for example, task generation features and license and billing services features (e.g., as may be associated with the customer 105 and/or the device 106).

In the example of FIG. 2, the simulation services component 144 is shown as including, for example, input builder features, orchestration and scheduling features, hypervisor features and one or more simulation engine features while the interactive 3D visualization component 148 is shown as including, for example, visualization features, stored data visualization features and one or more data representation engine features.

As an example, a cloud-based system can include customer input data interface and pre-processing interface features that include, for example, various sections related to login procedure, start menu and screens, digital rock and fluid database query formulation, tree structure for collecting simulation input data including numerical data introduction for digital rock and fluid properties as well as 2D and 3D digital images upload, simulation scenario definition and requirements.

As an example, a cloud-based system can include database services that include, for example, one or more database operational software application programs aimed to enter, track, gather and/or retrieve large quantities of digital rock, fluid and simulation scenario information related to a customer request and/or results of one or more simulation runs. In such an example, features may provide for automatic or semi-automatic semantic, machine learning and numerical attribute search for optimal data retrieval and handling. As an example, data mining operation can be implemented for extracting information from database sets and transform it into the valuable datasets for the further use.

As an example, a cloud-based system can include control services that include, for example, features for managing task generation and resource allocation for one or more simulation runs.

As an example, a cloud-based system can include simulation services that include, for example, features for optimized direct pore-scale hydrodynamic and petrophysical simulation (e.g., via one or more instances of the COREFLOW™ DHD simulator), which may be optimized for high performance computing operations, its supporting application components for a simulation input builder, orchestration and scheduler operations.

As an example, a cloud-based system can include visualization services that include, for example, features to perform 3D visualization of digital rock input data images, digital fluid data and simulation result representation including the comparative and sensitivity analysis of various simulation scenario runs. As an example, 3D visualization can be utilized to control acceptability of a simulation along computational time, for example, to highlight volumetric properties that may not be readily amendable to expression numerically and, for example, to analyze and record time-dependent 2D or 3D graphical output of one or more performed simulations.

As an example, a cloud-based system can include a customer result pre-processing and output interface that includes, for example, features for targeted applications for receiving information regarding one or more on-going simulations, characteristics and parameters of one or more on-going simulations, for example, including estimated time (e.g., to completion), resources utilized, quick look analysis and estimations, remote controlled interactive visualization of simulation results in the numerical data representation or multi-parameter and time-dependent 2D and 3D graphical outputs including also the stored database query representation and visualization.

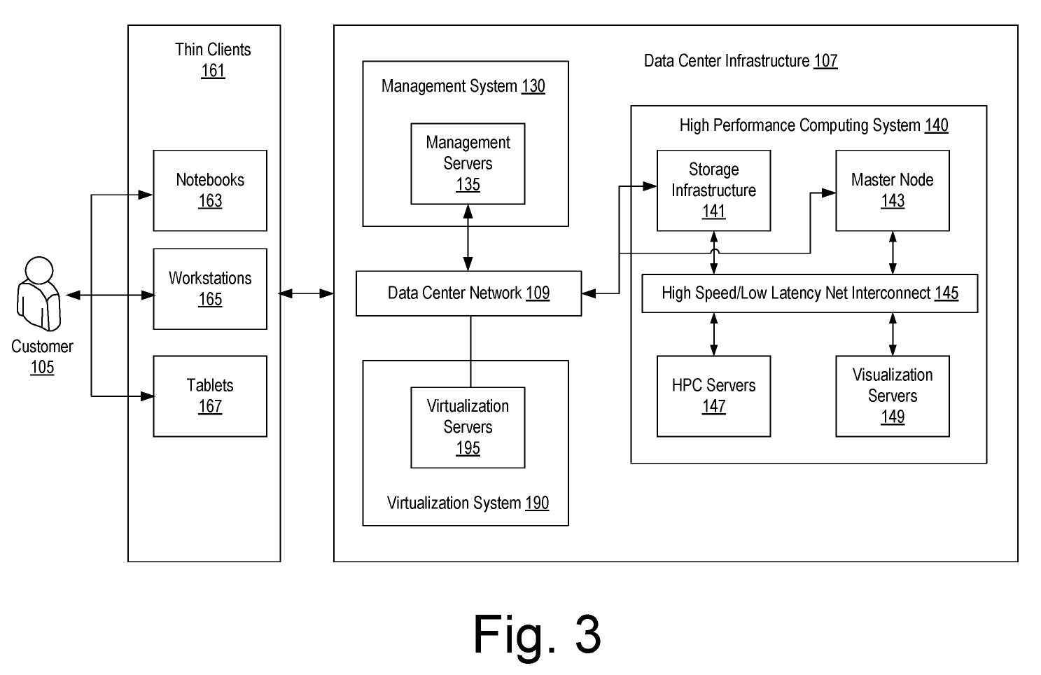

FIG. 3 shows an example of an architecture that includes a customer 105, a data center infrastructure 107 and one or more thin clients 161 that allow for interactions between the customer 105 and the data center infrastructure 107. As shown in FIG. 3, the data center infrastructure 107 can include a data center network 109. The data center network 109 can be an internal network where one or more components expose components operatively coupled to the internal network optionally, for example, with implementation of one or more of security, load balancing, etc. As shown in FIG. 3, the data center infrastructure 107 can include the management system components 130, the high performance computing system components 140 and virtualization system components 190, which include virtualization servers 195. As an example, virtualization may be implemented a part of a load balancing, service-based balancing, etc. approach where computing machines (e.g., virtual servers) may be instantiated responsive to demands placed on the data center infrastructure 107. As an example, the data center infrastructure 107 can optionally include one or more customer dedicated resources (e.g., dedicated to a particular customer) and/or one or more shared resources (e.g., suitable for use for a plurality of customers). Such options may correspond to security measures to assure that data is handled in a secure manner and/or in a manner specified by a customer and/or regulatory agency (e.g., government agency, etc.).

In the example of FIG. 3, the thin clients 161 can include notebooks 163, workstations 165, tablets 167 and/or one or more other types of thin client devices (e.g., mobile phones, etc.). Thin-client computing involves a client-server paradigm where a client device (e.g., end-user) sends control information (e.g., keyboard keys pressed, mouse pointer movement, touch screen display touches, gestures, etc.) to a server via a network and where the client device displays results of server mediated application execution results, progress, etc. on a display of the client device. In such an example, some instructions may be executed locally on the thin client device, for example, via browser application software and associated plug-ins, etc.; however, computation intensive instructions such as those of simulation are executed remote from the thin client device using one or more server mediated applications. As an example, rendering of information may be performed at a thin client device based on image data, graphics, vector graphics, etc., which may be generated server-side and transmitted to the thin client device. As an example, a thin client device may include one or more GPUs for rendering information to a display of the thin client device (e.g., a display operatively coupled to the thin client device).

As an example, a virtual reality system may be configured and utilized as a thin client device. In such an example, one or more users may participate in a virtual reality session, for example, to view renderings of simulation results for fluid flow in rock, a proppant pack, etc. As an example, a user interface may be a graphical user interface (GUI) that can be rendered to a display, via a virtual reality (VR) system, etc. As an example, a VR system may include one or more features of a VR system such as, for example, the HOLOLENS™ VR system marketed by Microsoft Corporation (Redmond, Washington). For example, a VR system may include goggles and/or one or more other types of wearables that can facilitate generation of and/or interaction with a virtual environment.

In the example of FIG. 3, the management system components 130 can include one or more management servers 135. The management system components 130 can include the servers 135 for running software in a design for managing cloud environments and operation. These operations can have the ability to manage a pool of computing resources and their allocation for simulations, data operation and visualization, provide a secure access to the users for forming the input information for simulation and receiving the output results, manage tracking between the database, simulation and visualization operations.

In the example of FIG. 3, the virtualization system components 190 can include virtual servers where some partition or segment of a physical machine or arrangement of physical machines makes it possible to run multiple operating systems and multiple applications on a server (e.g., at the same time). From the perspective of a user or application, a virtual machine can appear functionally as a physical machine. A virtual machine can include an operating system (e.g., referred to as a guest operating system) and at least one application program. As an example, one or more hypervisors may be utilized for purposes of managing one or more virtual machines (VMs).

In the example of FIG. 3, the high performance computing system components 140 include a storage infrastructure 141, a master node 143, a high speed/low latency network interconnect 145, one or more high performance computing (HPC) servers 147 and one or more visualization servers 149.

The HPCS components 140 can aim to provide information to one or more customers in an acceptable amount of time, which may be, for example, associated with an agreement, a license, a fee, etc. The HPCS components 140 can provide for performing simulations of pore-scale direct hydrodynamic and petrophysical simulations and provide for allowing customers to interactively analyze and visualize results.

As shown in FIG. 3, the HPCS components 140 include the storage infrastructure 141 and the master node 143 as operatively coupled to the data center network 109 and the high speed/low latency network interconnect 145. The performance computing servers (HPC servers) 147 and high performance visualization servers 149 are operatively connected through the high speed/low latency network interconnect (HPC network) 145. As mentioned, the storage infrastructure 141 is also connected to HPC network 145.

Amounts of data loaded, generated and stored during a simulation and visualization session of a customer can be in excess of a hundred gigabytes. Speed of used storage solution can influence overall performance of HPC system. As an example, an architecture can connect a storage system to a HPC network and use a HPC storage system such as, for example, the IBM General Parallel File System (GPFS), LUSTRE™ (Seagate Technology LLC, Cupertino, Calif.), etc.

The IBM GPFS is a high-performance clustered file system that can be deployed in shared-disk or shared-nothing distributed parallel modes. The IBM GPFS can allow data to be accessed over multiple computing devices concurrently. LUSTRE™ is a type of parallel distributed file system, generally used for large-scale cluster computing.

As an example, the storage infrastructure 141 can be available from one or more of the management servers 135 and one or more of the virtualization servers 195 optionally with lower requirements to network. In the example of FIG. 3, the master node 143 can automate scheduling, managing, monitoring, and reporting of HPC workloads. As an example, the master node 143 can include features to balance high utilization and throughput goals with competing workload priorities and customers' requirements. As an example, the master node 143 can initiate simulation and/or visualization tasks on available resources and monitor their status.

As to resource management, a master node or other component may implement one or more technologies. For example, consider Platform Load Sharing Facility (LSF) (e.g., IBM, Armonk, N.Y.), Moab (Adaptive Computing, Provo, Utah), etc. Platform Load Sharing Facility (LSF) is a workload management platform, job scheduler, for distributed HPC environments that may be used to execute batch jobs on networked UNIX™ (X/Open Company Ltd., Reading, United Kingdom) and WINDOWS™ (Microsoft Corporation, Redmond, Wash.) operating systems. The Moab Cluster Suite is a cluster workload management package that integrates the scheduling, managing, monitoring and reporting of cluster workloads.

As an example, the high performance computing (HPC) servers 147 and the high performance visualization servers (HPV) 149 can be configured to communicate with each other according to a message passing interface standard communication library over HPC network during parallel computing and processing of data. In FIG. 3, the HPC servers 147 and the HPV servers 149 can optionally load and save data from the storage infrastructure 141 via the HPC network 145. To provide maximal performance of computations, HPC servers may opt to forego virtualization such that programs run on “bare-metal” servers. As an example, the virtualization servers 195 may be optional and may be implemented for particular tasks that may be “high performance” or not (e.g., tasks that are less intensive than 3D flow simulation, etc.). As an example, one or more servers may be utilized in a parallel processing mode where tasks can be performed at least in part in parallel (e.g., consider simulations run in parallel for a customer's request).

As an example, the high performance computing system components 140 can include equipment such as, for example, HPC accelerators (e.g., NVIDIA™ TESLA™ GPU accelerators from NVIDIA Corporation, Santa Clara, Calif., INTEL™ XEON PHI coprocessors, Intel Corporation, Santa Clara, Calif., etc.).

As an example, the HPV servers 149 can allow for interactive remote visualization of 3D volumetric data from simulations. Where the HPC servers 147 include, for example, NVIDIA™ TESLA™ GPU accelerators they may be suitably used as HPV servers 149. In such an example, a server specification may include GPU capabilities such that one or more servers can be utilized for purposes of HPC and/or HPV. As an example, HPV servers can be base servers with, for example, one or more NVIDIA™ GPU cards (e.g., NVIDIA™ TESLA™ K40, NVIDIA™ QUADRO™ K6000, NVIDIA™ GRID K2, etc.). As an example, one or more servers can include virtualization that can support, for example, NVIDIA™ GRID vGPU technology.

As an example, a cloud-based system can include HPC and/or HPV servers that include one or more features of the HP PROLIANT™ SL250s Gen8 server with three NVIDIA™ TESLA™ K40 accelerators and can include a high speed and low latency network interconnect such as the FDR INFINIBAND™ interconnect (System I/O, Inc., Beaverton, Oreg.).

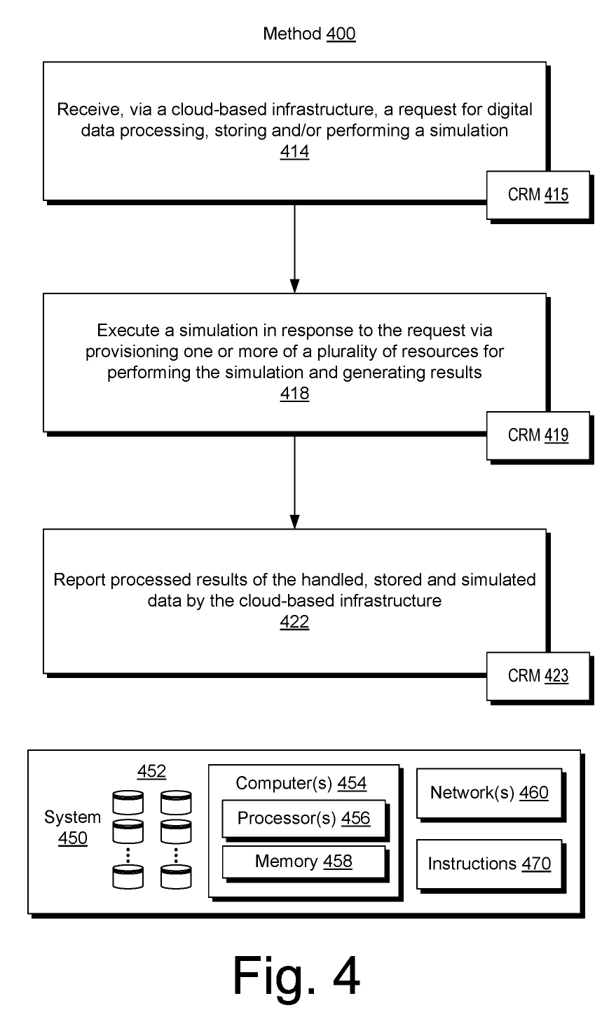

FIG. 4 shows an example of a method 400 for providing a cloud-based services (e.g., for digital information handling, storage and simulation associated to one or more digital rock workflows as accessible by a plurality of different entities) that includes a reception block 414 for receiving, via a cloud-based infrastructure, a request from a user device for digital data processing, storing and/or performing a simulation; an execution block 418 for executing a simulation in response to a request through provisioning one or more of a plurality of resources for performing the simulation and generating results; and a report block 422 for reporting to the user device processed results of the handled, stored and simulated data by the cloud-based infrastructure.

The method 400 is shown in FIG. 4 in association with various computer-readable media (CRM) blocks 415, 419 and 423. Such blocks generally include instructions suitable for execution by one or more processors (or cores) to instruct a computing device or system to perform one or more actions. While various blocks are shown, a single medium may be configured with instructions to allow for, at least in part, performance of various actions of the method 400. As an example, a computer-readable medium (CRM) may be a computer-readable storage medium that is non-transitory and not a carrier wave.

FIG. 4 also shows an example of a system 450 that includes one or more information storage devices 452, one or more computers 454, one or more networks 460 and instructions 470. As to the one or more computers 454, each computer may include one or more processors (e.g., or processing cores) 456 and memory 458 for storing instructions, for example, executable by at least one of the one or more processors. As an example, a computer may include one or more network interfaces (e.g., wired or wireless), one or more graphics cards, a display interface (e.g., wired or wireless), etc.

As an example, the instructions 470 (e.g., stored in memory) can be executable by one or more processors to instruct the system 450 to perform various actions. As an example, the system 450 may be configured such that the instructions 470 provide for establishing a framework or a portion thereof. As an example, one or more methods, techniques, etc. may be performed using the instructions 470 of FIG. 4.

As an example, a method can provide for fluid flow and petrophysical property evaluation of one or more materials such as, for example, reservoir rock, proppant, etc. As an example, one or more types of analyses can include pore-scale analysis in a material such as, for example, reservoir rock and/or proppant.

As an example, instructions can be part of an analysis framework such as, for example, the TECHLOG™ analysis framework and/or the COREFLOW™ framework. As an example, an OCEAN™ framework (Schlumberger Limited, Houston, Tex.) plug-in may be provided that allows interaction between the PETREL™ framework (Schlumberger Limited, Houston, Tex.) and the TECHLOG™ analysis framework and, for example, the VISAGE™ framework (Schlumberger Limited, Houston, Tex.), which can include instructions for modeling fracturing (e.g., hydraulic fracturing). As an example, the MANGROVE™ framework (Schlumberger Limited, Houston, Tex.) may be utilized for simulating behavior in a reservoir. As an example, a reservoir simulator framework such as the ECLIPSE™ framework (Schlumberger Limited, Houston, Tex.) or the INTERSECT™ framework (Schlumberger Limited, Houston, Tex.) may be utilized as part of a workflow. In such an example, information from digital rock simulation may be utilized (e.g., as to porosity, permeability, modeling, etc.).

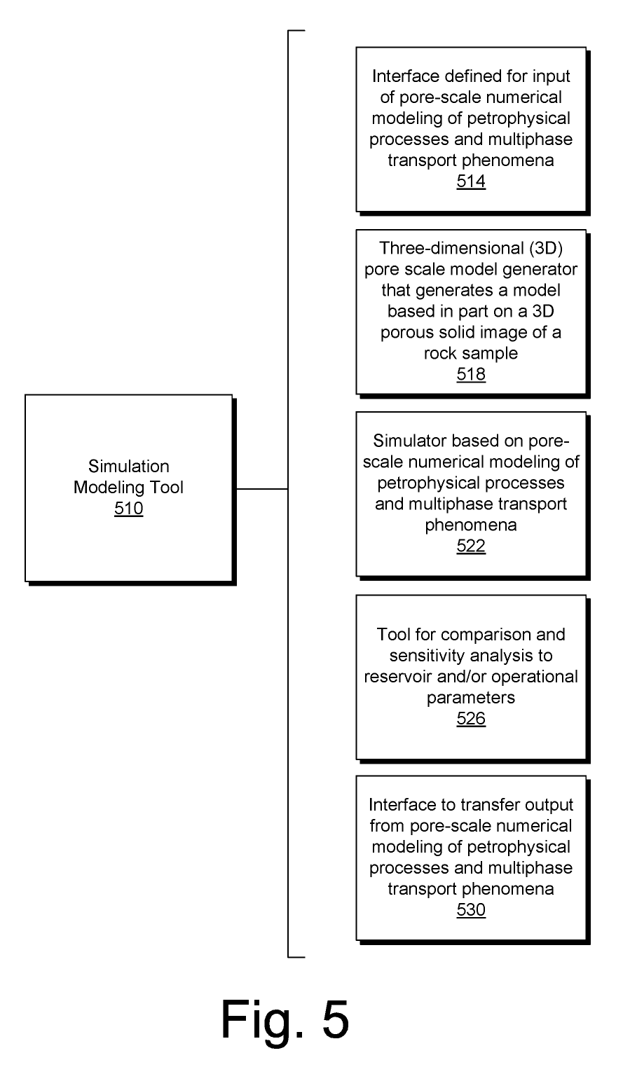

FIG. 5 shows an example of a simulation modeling tool 510 that can include various components such as, for example, an interface component 514 configured to define input for pore-scale numerical modelling of petrophysical processes and multiphase transport phenomena, a model generator component 518 for generating a three-dimensional (3D) pore scale model based at least in part on a 3D porous solid image of a rock sample, a simulator component 522 for simulation based on pore-scale numerical modelling of petrophysical processes and multiphase transport phenomena (e.g., for implementation as single or multiphase), a comparison and analysis component 526 as a tool for comparison and sensitivity analysis with respect to one or more reservoir and/or operational parameters, and an interface component 530 configured for transferring output from pore-scale numerical modelling of petrophysical processes and single and/or multiphase transport phenomena (e.g., to a client device, etc.).

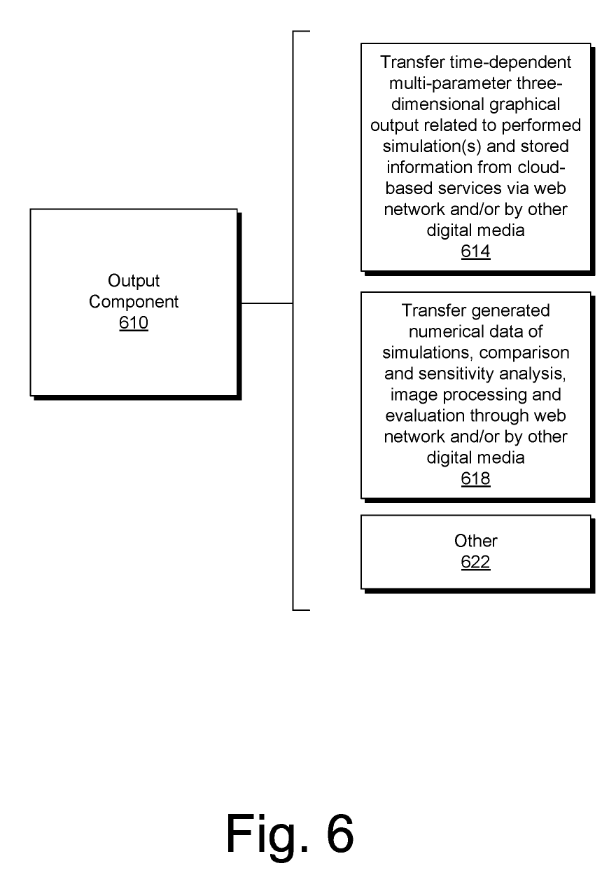

FIG. 6 shows an example of an output component 610 that can include various components such as, for example, a transfer component 614 for transferring time-dependent multi-parameter three-dimensional graphical output related to performed simulations and stored information from one or more cloud-based services to a client device via a Web network and/or via one or more other digital media; a transfer component 618 for transferring generated numerical data of simulations, comparison and sensitivity analysis, image processing and evaluation through web network and/or by other digital media; and an other block 622 for one or more other types of outputs.

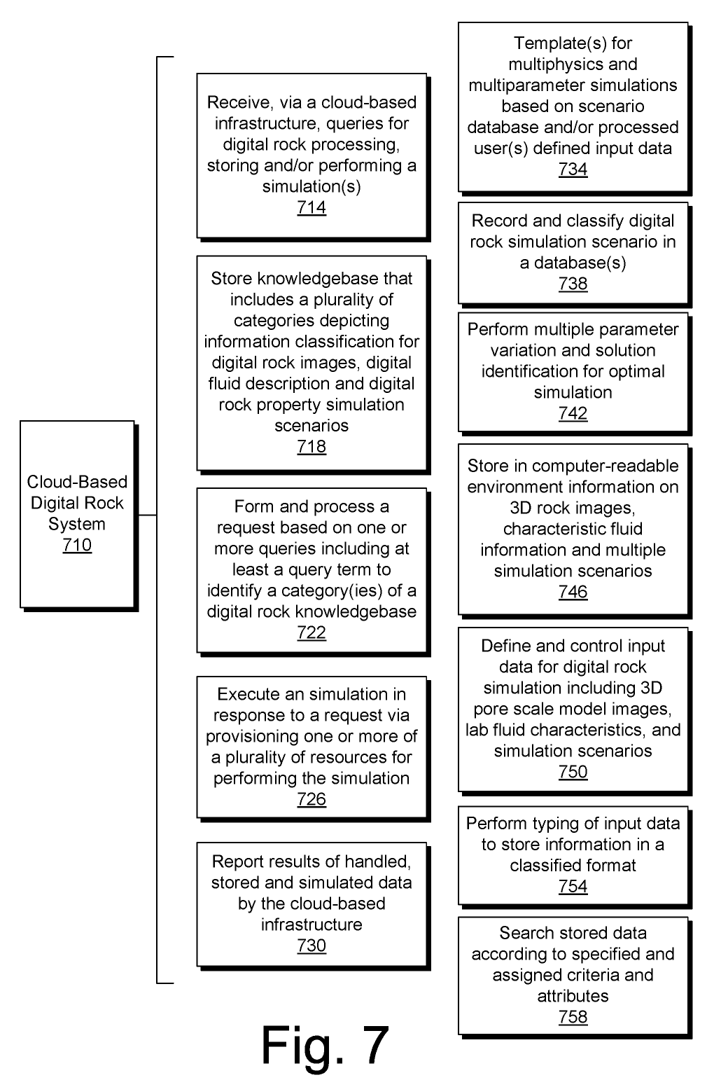

FIG. 7 shows an example of a cloud-based digital rock system 710 that can include various components such as, for example, a memory storage and a processor operatively connected to the memory through an internal network connection. In such an example, the system 710 can include components that can include executable instructions. Such components can include a reception component 714 for receiving, via a cloud-based infrastructure, queries from one or more client devices operatively coupled to the Internet for digital rock processing, storing and/or performing a simulation(s); a storage component 718 for storing a knowledgebase that includes a plurality of categories depicting information classification for digital rock images, digital fluid description and digital rock property simulation scenarios; a formation component 722 for forming and processing a request based on one or more queries from a client device including at least query term to identify one or more categories of a digital rock knowledgebase; an execution component 726 for executing a simulation in response to a request via provisioning one or more of a plurality of resources for performing the simulation; and a report component 730 for reporting to a client device processed results of the handled, stored and simulated data by the cloud-based infrastructure.

In the example of FIG. 7, the system 710 may also include a template component 734 for one or more templates for multiphysics and multiparameter simulations based on the scenario database and/or processed user defined input data; a record component 738 for recording and classifying digital rock simulation scenarios in a database or databases; a performance component 742 for performing multiple parameter variation and solution identification for optimal simulation; a storage component 746 for storing in a computer-readable environment information on 3D rock images, characteristic fluid information and multiple simulation scenarios; a definition component 750 for defining and controlling input data for digital rock simulation including 3D pore scale model images, lab fluid characteristics, simulation scenarios; a performance component 754 for performing typing of input data to store information in a classified format; and a search component 758 for searching in the stored data according to one or more specified and/or assigned criteria and/or attributes.

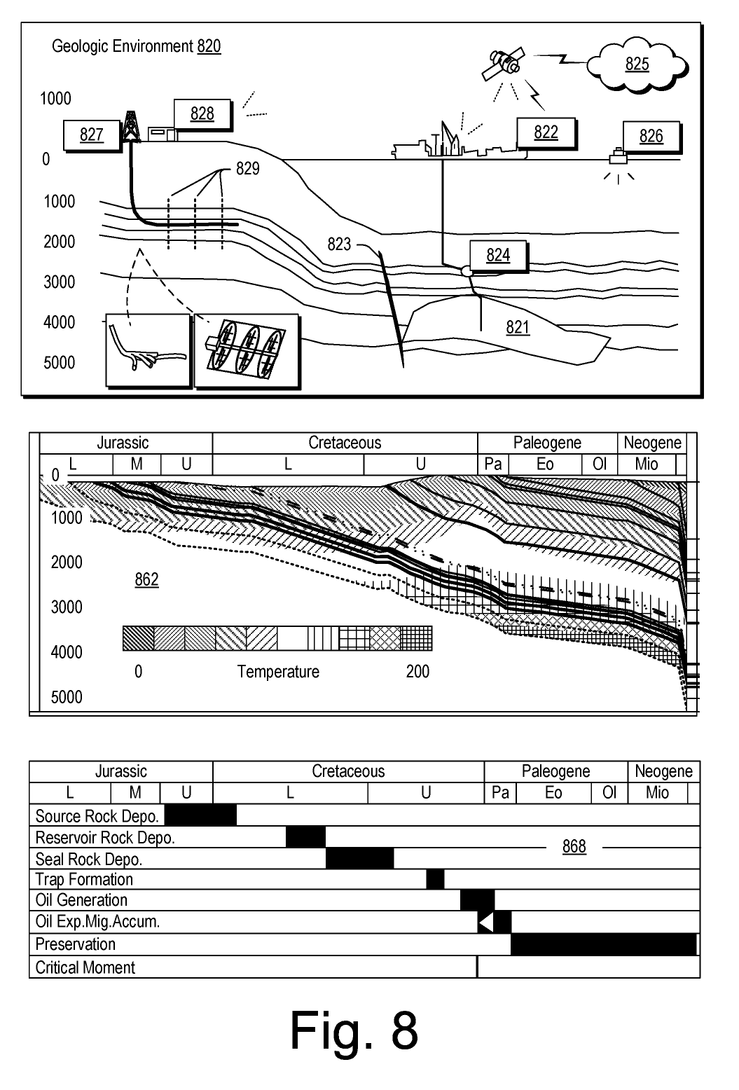

FIG. 8 shows an example of a geologic environment 820. In FIG. 8, the geologic environment 820 may be a sedimentary basin that includes layers (e.g., stratification) that include a reservoir 821 and that may be, for example, intersected by a fault 823 (e.g., or faults). As an example, the geologic environment 820 may be outfitted with any of a variety of sensors, detectors, actuators, etc. For example, equipment 822 may include communication circuitry to receive and to transmit information with respect to one or more networks 825. Such information may include information associated with downhole equipment 824, which may be equipment to acquire information, to assist with resource recovery, etc. Other equipment 826 may be located remote from a well site and include sensing, detecting, emitting or other circuitry. Such equipment may include storage and communication circuitry to store and to communicate data, instructions, etc. As an example, one or more pieces of equipment may provide for measurement, collection, communication, storage, analysis, etc. of data (e.g., for one or more produced resources, etc.). As an example, one or more satellites may be provided for purposes of communications, data acquisition, etc. For example, FIG. 8 shows a satellite in communication with the network 825 that may be configured for communications, noting that the satellite may additionally or alternatively include circuitry for imagery (e.g., spatial, spectral, temporal, radiometric, etc.).

FIG. 8 also shows the geologic environment 820 as optionally including equipment 827 and 828 associated with a well that includes a substantially horizontal portion (e.g., or portions; see, e.g., the enlarged view of a well with lateral portions) that may intersect with one or more fractures 829 (see, e.g., the enlarged view with fractures that can define a drainage area). For example, consider a well in a shale formation that may include natural fractures, artificial fractures (e.g., hydraulic fractures) or a combination of natural and artificial fractures. As an example, a well may be drilled for a reservoir that is laterally extensive. In such an example, lateral variations in properties, stresses, etc. may exist where an assessment of such variations may assist with planning, operations, etc. to develop the reservoir (e.g., via fracturing, injecting, extracting, etc.). As an example, the equipment 827 and/or 828 may include components, a system, systems, etc. for fracturing, seismic sensing, analysis of seismic data, assessment of one or more fractures, injection, production, etc. As an example, the equipment 827 and/or 828 may provide for measurement (e.g., temperature, pressure, etc.), collection, communication, storage, analysis, etc. of data such as, for example, production data (e.g., for one or more produced resources). As an example, one or more satellites may be provided for purposes of communications, data acquisition, etc.

FIG. 8 also shows a plot 862 of temperature with respect to depth and time and a petroleum systems elements (PSE) chart 868. The information in the plots 862 and the chart 868 can be based on various types of measurements and one or more types of models, for example, one or more models suitable for one or more types of simulations.

With respect to petroleum system elements (PSE), temporal aspects can include, for example, depositional or formation ages, “critical” moment, and preservation time. In a PSE analysis, a “critical” moment is the time that best depicts the generation-migration-accumulation of hydrocarbons in a petroleum system and preservation time of a petroleum system begins immediately after the generation-migration-accumulation process occurs and may extend to the present day.

A PSE chart may be arranged according to an ideal or successful order of events. For example, the source rock could be generated and expel hydrocarbons once the trap is formed. In an example embodiment, a PSE chart may serve as a basis for risk analysis or be transformed into a risk chart, for example, to better evaluate a play or prospect.

Various types of frameworks can receive information, analyze information and generate results for a geologic environment and/or one or more related operations. As to some examples of frameworks, consider the COREFLOW™ framework (Schlumberger Limited, Houston, Tex.), PETREL™ framework (Schlumberger Limited, Houston, Tex.), which provides for interpretation of seismic data, model building, etc., the ECLIPSE™ reservoir simulator (Schlumberger Limited, Houston, Tex.), the INTERSECT™ reservoir simulator (Schlumberger Limited, Houston, Tex.), the VISAGE™ geomechanics simulator (Schlumberger Limited, Houston, Tex.), the PETROMOD™ petroleum systems simulator (Schlumberger Limited, Houston, Tex.), the PIPESIM™ network simulator (Schlumberger Limited, Houston, Tex.), the TECHLOG™ framework, etc.

The ECLIPSE™ simulator includes numerical solvers that may provide simulation results such as, for example, results that may predict dynamic behavior for one or more types of reservoirs. The VISAGE™ geomechanics simulator includes finite element numerical solvers that may provide simulation results such as, for example, results as to compaction and subsidence of a geologic environment, well and completion integrity in a geologic environment, cap-rock and fault-seal integrity in a geologic environment, fracture behavior in a geologic environment, thermal recovery in a geologic environment, CO₂ disposal, etc. The PETROMOD™ simulator includes finite element numerical solvers that may provide simulation results such as, for example, results as to structural evolution, temperature, and pressure history and as to effects of such factors on generation, migration, accumulation, and loss of oil and gas in a petroleum system through geologic time. Such a simulator can provide properties such as, for example, gas/oil ratios (GOR) and API gravities, which may be analyzed, understood, and predicted as to a geologic environment. The PIPESIM™ simulator includes solvers that may provide simulation results such as, for example, multiphase flow results (e.g., from a reservoir to a wellhead and beyond, etc.), flowline and surface facility performance, etc. The PIPESIM™ simulator may be integrated, for example, with the AVOCET™ production operations framework (Schlumberger Limited, Houston Tex.). As an example, a reservoir or reservoirs may be simulated with respect to one or more enhanced recovery techniques (e.g., consider a thermal process such as SAGD, etc.). As an example, information acquired by a tool or tools may be analyzed using a framework such as the TECHLOG™ framework (Schlumberger Limited, Houston, Tex.). Data exchange between frameworks can facilitate construction of models, analysis of data (e.g., PETROMOD™ framework data analyzed using PETREL™ framework capabilities), and coupling of workflows.

One or more frameworks can include interfaces for receiving information that can include measurements and/or information based on measurements. As an example, one or more simulators may generate results that are based at least in part on measurements. As an example, a framework and/or a simulator may implement one or more methods for estimation of formation pressure and/or formation temperature using a combination of borehole logs (e.g., measurements).

Geologic formations such as in the geologic environment 820 include rock, which may be characterized by, for example, porosity values and by permeability values. Porosity may be defined as a percentage of volume occupied by pores, void space, volume within rock that can include fluid, etc. Permeability may be defined as an ability to transmit fluid, measurement of an ability to transmit fluid, etc.

As an example, rock may include clastic material, carbonate material and/or other type of material. As an example, clastic material may be material that includes broken fragments derived from preexisting rocks and transported elsewhere and redeposited before forming another rock. Examples of clastic sedimentary rocks include siliciclastic rocks such as conglomerate, sandstone, siltstone and shale. As an example, carbonate material may include calcite (CaCO₃), aragonite (CaCO₃) and/or dolomite (CaMg(CO₃)₂), which may replace calcite during a process known as dolomitization. Limestone, dolostone or dolomite, and chalk are some examples of carbonate rocks. As an example, carbonate material may be formed through processes of precipitation or the activity of organisms (e.g., coral, algae, etc.). Carbonates may form in shallow and deep marine settings, evaporitic basins, lakes, windy deserts, etc. Carbonate material deposits may serve as hydrocarbon reservoir rocks, for example, where porosity may have been enhanced through dissolution. Fractures can increase permeability in carbonate material deposits.

The term “effective porosity” may refer to interconnected pore volume in rock, for example, that may contribute to fluid flow in a formation. As effective porosity aims to exclude isolated pores, effective porosity may be less than total porosity. As an example, a shale formation may have relatively high total porosity yet relatively low permeability due to how shale is structured within the formation.

As an example, shale may be formed by consolidation of clay- and silt-sized particles into thin, relatively impermeable layers. In such an example, the layers may be laterally extensive and form caprock. Caprock may be defined as relatively impermeable rock that forms a barrier or seal with respect to reservoir rock such that fluid does not readily migrate beyond the reservoir rock. As an example, the permeability of caprock capable of retaining fluids through geologic time may be of the order of about 10⁻⁶ to about 10⁻⁸ D (darcies).

The term “shale” may refer to one or more types of shales that may be characterized, for example, based on lithology, etc. In shale gas formations, gas storage and flow may be related to combinations of different geophysical processes. For example, regarding storage, natural gas may be stored as compressed gas in pores and fractures, as adsorbed gas (e.g., adsorbed onto organic matter), and as soluble gas in solid organic materials.

Gas migration and production processes in gas shale sediments can occur, for example, at different physical scales. As an example, production in a newly drilled wellbore may be via large pores through a fracture network and then later in time via smaller pores. As an example, during reservoir depletion, thermodynamic equilibrium among kerogen, clay and the gas phase in pores can change, for example, where gas begins to desorb from kerogen exposed to a pore network.

Sedimentary organic matter tends to have a high sorption capacity for hydrocarbons (e.g., adsorption and absorption processes). Such capacity may depend on factors such as, for example, organic matter type, thermal maturity (e.g., high maturity may improve retention) and organic matter chemical composition. As an example, a model may characterize a formation such that a higher total organic content corresponds to a higher sorption capacity.

With respect to a formation that includes hydrocarbons (e.g., a hydrocarbon reservoir), its hydrocarbon producing potential may depend on various factors such as, for example, thickness and extent, organic content, thermal maturity, depth and pressure, fluid saturations, permeability, etc. As an example, a formation that includes gas (e.g., a gas reservoir) may include nanodarcy matrix permeability (e.g., of the order of 10⁻⁹ D) and narrow, calcite-sealed natural fractures. In such an example, technologies such as stimulation treatment may be applied in an effort to produce gas from the formation, for example, to create new, artificial fractures, to stimulate existing natural fractures (e.g., reactivate calcite-sealed natural fractures), etc. (see, e.g., the one or more fractures 829 in the geologic environment 820 of FIG. 8).

Material in a geologic environment may vary by, for example, one or more of mineralogical characteristics, formation grain sizes, organic contents, rock fissility, etc. Attention to such factors may aid in designing an appropriate stimulation treatment or one or more other operations. An evaluation process may include well construction (e.g., drilling one or more vertical, horizontal or deviated wells), sample analysis (e.g., for geomechanical and geochemical properties), open-hole logs (e.g., petrophysical log models) and post-fracture evaluation (e.g., production logs). Effectiveness of a stimulation treatment (e.g., treatments, stages of treatments, etc.), may determine flow mechanism(s), well performance results, etc.

As an example, a stimulation treatment may include pumping fluid into a formation via a wellbore at pressure and rate sufficient to cause a fracture to open. Such a fracture may be vertical and include wings that extend away from the wellbore, for example, in opposing directions according to natural stresses within the formation. As an example, proppant (e.g., sand, etc.) may be mixed with treatment fluid to deposit the proppant in the generated fractures in an effort to maintain fracture width over at least a portion of a generated fracture. For example, a generated fracture may have a length of about 500 ft (e.g., about 150 m) extending from a wellbore where proppant maintains a desirable fracture width over about the first 250 ft (e.g., about 75 m) of the generated fracture.

In a stimulated gas formation, fracturing may be applied over or within a region deemed a “drainage area” (e.g., consider at least one well with at least one artificial fracture), for example, according to a development plan. In such a formation, gas pressure (e.g., within the formation's “matrix”) may be higher than in generated fractures of the drainage area such that gas flows from the matrix to the generated fractures and onto a wellbore. During production of the gas, gas pressure in a drainage area tends to decrease (e.g., decreasing the driving force for fluid flow, for example, per Darcy's law, Navier-Stokes equations, etc.). As an example, gas production from a drainage area may continue for decades; however, the predictability of decades long production (e.g., a production forecast) can depend on many factors, some of which may be uncertain (e.g., unknown, unknowable, estimated with probability bounds, etc.).

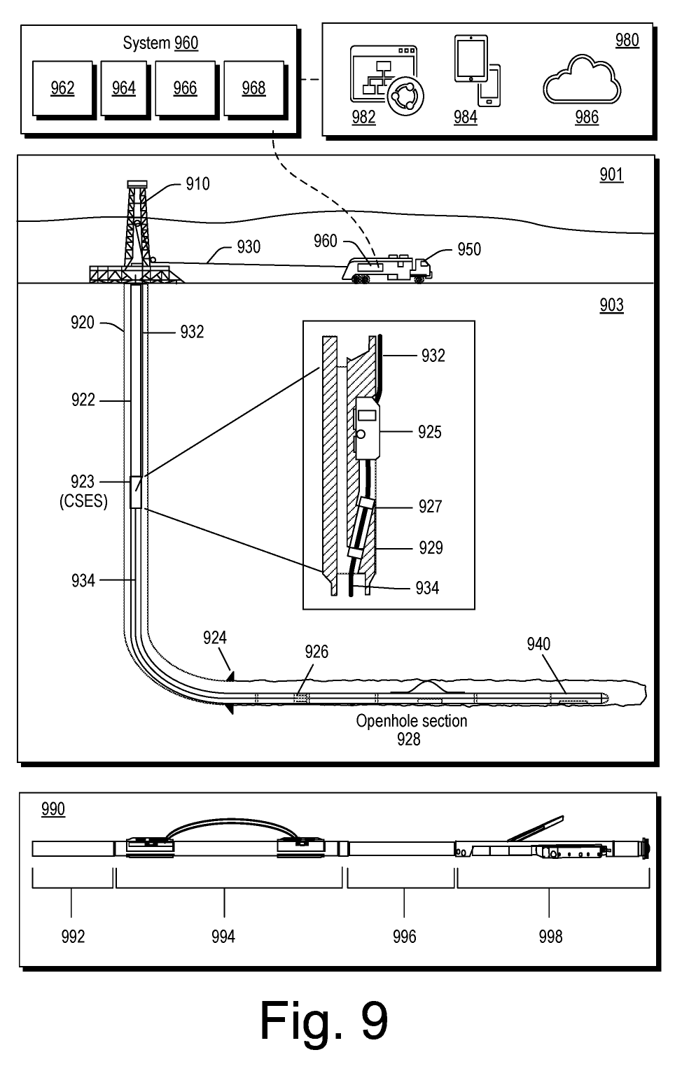

FIG. 9 shows an example of an environment 901 that includes a subterranean portion 903 where a rig 910 is positioned at a surface location above a bore 920. In the example of FIG. 9, various wirelines services equipment can be operated to perform one or more wirelines services including, for example, acquisition of data from one or more positions within the bore 920.

In the example of FIG. 9, the bore 920 includes drillpipe 922, a casing shoe, a cable side entry sub (CSES) 923, a wet-connector adaptor 926 and an openhole section 928. As an example, the bore 920 can be a vertical bore or a deviated bore where one or more portions of the bore may be vertical and one or more portions of the bore may be deviated, including substantially horizontal.

In the example of FIG. 9, the CSES 923 includes a cable clamp 925, a packoff seal assembly 927 and a check valve 929. These components can provide for insertion of a logging cable 930 that includes a portion 932 that runs outside the drillpipe 922 to be inserted into the drillpipe 922 such that at least a portion 934 of the logging cable runs inside the drillpipe 922. In the example of FIG. 9, the logging cable 930 runs past the wet-connect adaptor 926 and into the openhole section 928 to a logging string 940.

As shown in the example of FIG. 9, a logging truck 950 (e.g., a wireline services vehicle) can deploy the wireline 930 under control of a system 960. As shown in the example of FIG. 9, the system 960 can include one or more processors 962, memory 964 operatively coupled to at least one of the one or more processors 962, instructions 966 that can be, for example, stored in the memory 964, and one or more interfaces 968. As an example, the system 960 can include one or more processor-readable media that include processor-executable instructions executable by at least one of the one or more processors 962 to cause the system 960 to control one or more aspects of equipment of the logging string 940 and/or the logging truck 950. In such an example, the memory 964 can be or include the one or more processor-readable media where the processor-executable instructions can be or include instructions. As an example, a processor-readable medium can be a computer-readable storage medium that is not a signal and that is not a carrier wave.

As an example, the system 960 can be operatively coupled to a client layer 980. In the example of FIG. 9, the client layer 980 can include features that allow for access and interactions via one or more private networks 982, one or more mobile platforms and/or mobile networks 984 and via the “cloud” 986, which may be considered to include distributed equipment that forms a network such as a network of networks. As an example, the system 960 can include circuitry to establish a plurality of connections (e.g., sessions). As an example, connections may be via one or more types of networks. As an example, connections may be client-server types of connections where the system 960 operates as a server in a client-server architecture. For example, clients may log-in to the system 960 where multiple clients may be handled, optionally simultaneously.

FIG. 9 also shows an example of a toolstring 990 that can include various assemblies. For example, the toolstring 990 can include a hostile-environment natural gamma ray sonde (HNGS) assembly 992, an accelerator porosity sonde (APS) assembly 994, an integrated porosity lithology (IPL) cartridge assembly 996 and a litho-density sonde (LDS) assembly 998. As an example, the toolstring 990 may be an integrated porosity lithology (IPL) system such as, for example, the IPL system marketed by Schlumberger Limited, Houston, Tex. While some examples of tools are mentioned with respect to the toolstring 990, a toolstring may include one or more other types of tools and may be suitable for deployment and use with one or more wireline services systems.

As an example, a toolstring can include circuitry, which may include one or more controllers, memory, etc. As an example, a controller may be a microcontroller (e.g., an ARM chip, etc.), a processor, an ASIC, etc. As an example, a controller may operate via instructions stored in memory (e.g., firmware instructions, software instructions, RISC instructions, etc.). As an example, circuitry may be included in a cartridge. As an example, one or more assemblies may include interfaces, for example, for communication of information. As an example, one or more assemblies may include memory, for example, as a storage device that may store one or more of data and instructions. As an example, a method may be implemented in part via instructions that may be executable by circuitry (e.g., a controller, microcontroller, processor, etc.).

Wireline services can include deployment of one or more tools in a bore in a geologic environment, for example, as drilled via a rig. Wireline services can include acquiring petrophysical measurements that can, for example, help to determine petrophysical properties of a reservoir, its fluid contents, etc. Some examples of wireline services tools include a lithology scanner spectrometer (e.g., to measure elements and quantitatively determine total organic carbon (TOC) in a wide variety of formations), a dielectric scanner (e.g., to measure water volume and rock textural information to determine hydrocarbon volume, whether in carbonates, shaly or laminated sands, or heavy oil reservoirs), a magnetic resonance scanner (e.g., to acquire NMR measurement of porosity, permeability, and fluid volumes), an Rt scanner (e.g., to acquire resistivity measurements germane to formation dip, anisotropy, beds, etc.), a sonic scanner acoustic scanning platform (e.g., to understand a reservoir stress regime and anisotropy through 3D acoustic measurements made axially, azimuthally, and/or radially), an analysis behind casing tool, (e.g., well log data—including the collection of fluid samples—in cased holes to find bypassed pay, etc.), etc.

As mentioned, wireline services can include conveying equipment in a bore of a geologic environment. Conveyance can be performed by a crew in a hands-on manner to account for bore characteristics, particularly bore geometries.

As an example, a tool may be configured to acquire electrical borehole images. As an example, the fullbore Formation Microlmager (FMI) tool (Schlumberger Limited, Houston, Tex.) can acquire borehole image data. A data acquisition sequence for such a tool can include running the tool into a borehole with acquisition pads closed, opening and pressing the pads against a wall of the borehole, delivering electrical current into the material defining the borehole while translating the tool in the borehole, and sensing current remotely, which is altered by interactions with the material.

Analysis of information may reveal features such as, for example, vugs, dissolution planes (e.g., dissolution along bedding planes), stress-related features, dip events, etc. As an example, a tool may acquire information that may help to characterize a reservoir, optionally a fractured reservoir where fractures may be natural and/or artificial (e.g., hydraulic fractures).

As an example, a method may be performed in real time or near real time where a tool or toolstring is moved in a borehole. As an example, where a field includes a plurality of boreholes, borehole data from one or more tools or toolstrings may be inverted in real time or near real time for formation pressure and formation temperature. In such an example, formation pressure and/or formation temperature may be rendered to a display for the boreholes, for example, with locations in a formation (e.g., as to depth, which may be measured depth). In such an example, formation pressures and/or formation temperatures may be linked to generate a line or a surface in a formation, which may, for example, be associated with a layer, which may be laterally extensive and span a range of depths.

A subterranean formation is an underground geological region. An underground geological region is a geographic area that exists below land or ocean. In one or more embodiments, the underground geological region includes the subsurface formation in which a borehole is or may be drilled and any subsurface region that may affect the drilling of the borehole, such as because of stresses and strains existing in the subsurface region. In other words, the underground geological region may include the area immediately surrounding a borehole or where a borehole may be drilled, but also any area that affects or may affect the borehole or where the borehole may be drilled.

A subterranean formation may include several geological structures such as, for example, a sandstone layer, a limestone layer, a shale layer, and a sand layer. A fault line may extend through such a formation. Various survey tools and/or data acquisition tools can be adapted to measure a formation and detect characteristics of geological structures of the formation.

As an example, a surface unit can be operatively coupled to a field management tool and/or a wellsite system. A wellsite system may be adapted for measuring downhole properties using logging-while-drilling (“LWD”) tools to obtain well logs and for obtaining core samples. A surface unit may be located at a wellsite system and/or at a remote location. A surface unit may send command signals to field equipment in response to data received, for example, to control and/or optimize various field operations.

During various oilfield operations, data can be collected for analysis and/or monitoring of the oilfield operations. Such data may include, for example, geological information concerning subterranean formations, descriptions of tools and equipment, and historical and/or other data. Static data relates to, for example, formation structure and geological stratigraphy that define the geological structures of the subterranean formation. Static data may also include data about the wellbore, such as inside diameters, outside diameters, and depths. Dynamic data relates to, for example, fluids flowing through the geologic structures of the subterranean formation over time. The dynamic data may include, for example, pressures, fluid compositions (e.g. gas-oil ratio, water cut, and/or other fluid compositional information), states of various equipment, and other information.

The static and dynamic data collected from the wellbore and the oilfield may be used to create and update a three-dimensional model of the subsurface formations. Additionally, static and dynamic data from other wellbores or oilfields may be used to create and update the three-dimensional model. Hardware sensors, core sampling, and well logging techniques may be used to collect the data. Other static measurements may be gathered using downhole measurements, such as core sampling and well logging techniques. Well logging involves deployment of a downhole tool into the wellbore to collect various downhole measurements, such as density, resistivity, etc., at various depths. Such well logging may be performed using, for example, a drilling tool and/or a wireline tool, or sensors located on downhole production equipment. Once the well is formed and completed, fluid can flow using production tubing and other completion equipment. As an example, for a production well, as fluid passes to the surface, various dynamic measurements, such as fluid flow rates, pressure, and composition may be monitored. These parameters may be used to determine various characteristics of the subterranean formation.

Data received by a surface unit may be communicatively coupled to a field management tool, which may be configured to analyze, model, control, optimize, or perform other management tasks of the aforementioned field operations based at least in part on the data provided from the surface unit.

Sensors can be located about a wellsite to collect data, possibly in real time, concerning the operation of the wellsite, as well as conditions at the wellsite. The sensors may also have features or capabilities, of monitors, such as cameras, to provide pictures of the operation (e.g., drones, fixed cameras, etc.). Surface sensors or gauges may be deployed about the surface systems to provide information about the surface unit, such as standpipe pressure, hook load, depth, surface torque, and/or rotary speed, among others. Downhole sensors or gauges are disposed about the drilling tool and/or wellbore to provide information about downhole conditions, such as wellbore pressure, weight-on-bit, torque-on-bit, direction, inclination, collar rotary speed, tool temperature, annular temperature, and toolface, among others. For example, sensors may include one or more of a camera, a pressure sensor, a temperature sensor, a flow rate sensor, a vibration sensor, a current sensor, a voltage sensor, a resistance sensor, a gesture detection sensor or device, a voice actuated or recognition device or sensor, a seismic sensor, or one or more other suitable sensors. Information collected by sensors and/or cameras (e.g., or other on-site imaging equipment such as, for example, X-ray microCT) may be conveyed to one or more parts of a drilling system and/or a surface unit for on-site and/or remote processing.

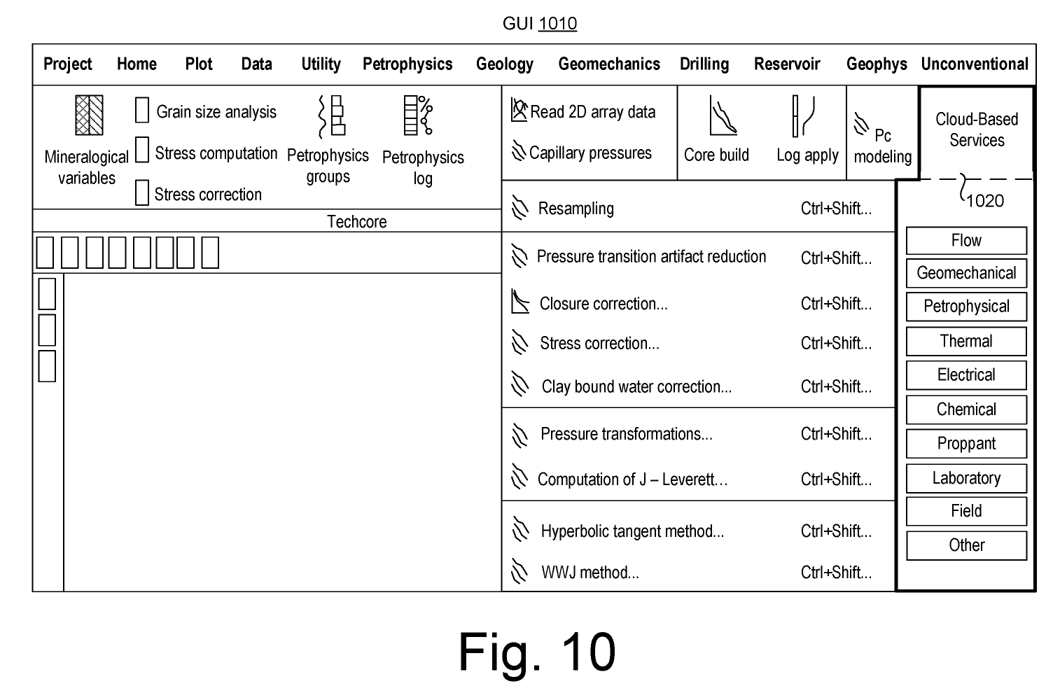

FIG. 10 shows an example of a graphical user interface (GUI) 1010 that may be part of a cloud-based system executed using one or more processors, memory accessibly the at least one of the one or more processors, etc. As an example, the GUI 1010 may be part of a framework such as the TECHLOG™ framework, which may be operatively coupled to the COREFLOW™ framework.

As shown in FIG. 10, the GUI 1010 may include various options associated with material analyses, which, in turn, may aid in characterizing materials for use in one or more operations. As an example, a workflow may include one or more worksteps associated with one or more graphical controls of the GUI 1010. As an example, a workflow may include performing one or more field operations. As an example, a field operation may include acquiring one or more samples, drilling, injecting fluid, producing fluid, etc. As an example, a field operation may depend in part on results of an analysis of a sample (e.g., core, proppant, etc.) and optionally one or more fluids and/or one or more chemicals.

In FIG. 10, the GUI 1010 includes a graphical control 1020 for access to cloud-based services. In such an example, the graphical control 1020 may be selected to perform one or more analyses, which can include, for example, digital rock simulation. For example, the customer 105 of FIG. 1 may utilize the computing device 106 to interact with the GUI 1010 as rendered to a display of the computing device 106 to select the graphical control 1020 to instruct a cloud-based system to perform a simulation at least in part via the simulation modeling tool 510 of FIG. 5.

As an example, the GUI 1010 may be operatively coupled to equipment at a field site as in, for example, FIG. 8 or FIG. 9. As mentioned, the system 960 can be operatively coupled to the cloud 986. In such an example, information generated via cloud-based digital rock simulation may be based at least in part on information acquired via one or more tools (e.g., logging tools, imaging, tools, etc.) and/or information generated via cloud-based digital rock simulation may be utilized to set or adjust one or more parameters associated with one or more field operations. For example, one or more acquisition parameters of a logging tool may be adjusted (e.g., tuned) based at least in part on digital rock simulation results. As an example, a feedback loop may be established where information is acquired on-site (in the field) and where digital rock simulation is performed remotely using cloud-based resources. Results of the digital rock simulation can be based at least in part on information acquired on-site (in the field) and such results can be transmitted to the on-site equipment (field equipment), which may assist with acquisition of additional information. In such an example, a method can include converging iteratively between field acquired data and digital rock simulation results. Such a method may facilitate characterizing rock such as, for example, reservoir rock. Characterized rock can be a basis for making one or more decisions as to one or more phases of field operations as to development of a reservoir or reservoirs (e.g., for production of fluid or fluids).

As an example, the logging truck 950 can be represented in a cloud-based infrastructure that hosts a digital rock analysis and database services system where requests may be made via a client device or client devices in the logging truck 950 (e.g., associated with the logging truck). In such an example, a learning algorithm of the cloud-based infrastructure can learn about how the logging truck 950 interacts with services of the system such that analyses may be enhanced such as being made more efficient and/or more accurate. For example, where the logging truck 950 is to perform logging operations in a plurality of boreholes in a field, the digital rock analysis and database services system can learn as data and/or service requests are made as associated with a plurality of individual boreholes. In such an example, a digital, image-based model of rock material can be enhanced (e.g., fine-tuned, etc.) and optionally utilized to re-perform one or more prior analyses (e.g., as to earlier assessed boreholes) to enhance analysis results based on one or more later analyses. In such an example, a digital, image-based model or models of rock material in the field may be generated as a master model or master models at the completion of logging operations in the field. Such a model or models may be associated with a single logging truck or optionally a plurality of logging trucks. A master model or models may be utilized by a cloud-based digital rock analysis and database services system for one or more purposes associated with development of the field (e.g., drilling, stimulation, injection, production, etc.).

While a logging truck as field equipment is mentioned, laboratory equipment can be represented in a cloud-based infrastructure that hosts a digital rock analysis and database services system. As an example, operation of the equipment may automatically transmit information to the cloud-based infrastructure, which, in turn, may automatically provision resources for one or more purposes. For example, resources may be provisioned to analyze measurements from the equipment and/or to store measurements from the equipment. As an example, a laboratory can include various types of equipment operatively coupled to a cloud-based infrastructure that hosts a digital rock analysis and database services system via one or more network interfaces. Such a system may be “remote” and provide analysis results locally to one or more client devices in the laboratory and/or to one or more remote client devices (e.g., via a results distribution list, etc.). As an example, a system may track laboratory equipment and/or measurements and optionally assess equipment status. As an example, a system may assess quality of results of equipment, track order of samples being analyzed, track pending samples to be analyzed, transmit recommendations as to analysis techniques, etc. As an example, a system can include provisioning and de-provisioning cloud-based resources (e.g., virtual machines, processing cores, memory, etc.) in an automated manner based on utilization of equipment in a laboratory or equipment in laboratories, optionally based on one or more requests for analysis of laboratory data, etc., which may be generated automatically responsive to use of equipment.

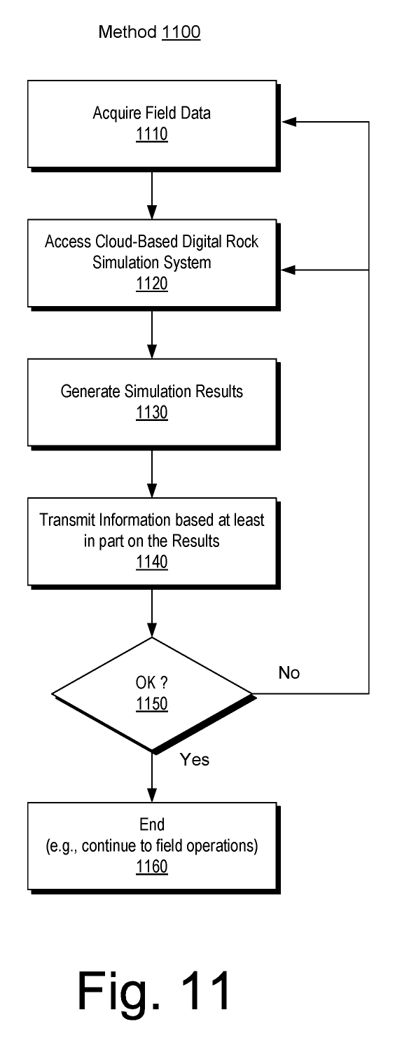

FIG. 11 shows an example of a method 1100 that includes an acquisition block 1110 for acquiring field data, an access block 1120 for accessing a cloud-based digital rock simulation system, a generation block 1130 for generating simulation results, a transmit block 1140 for transmitting information based at least in part on the results, a decision block 1150 for deciding whether the information indicates that the results and/or the field data are acceptable and a termination block 1160 for terminating the method 1100 where the results and/or the field data are acceptable. As shown, where the results and/or the field data are deemed not acceptable, the method 1100 may continue to the acquisition block 1110 and/or, for example, to the access block 1120 to acquire additional field data and/or to adjust one or more parameters associated with digital rock simulation (e.g., fluid flow simulation in a digital rock model that is based on imagery of actual rock).

While the method 1100 includes acquisition of field data, such a method may be implemented with respect to acquisition of laboratory data and/or with respect to acquisition of field data and laboratory data. In such examples, field and/or laboratory equipment can be utilized to access a cloud-based digital rock simulation system where one or more decisions may be made to acquire additional field data and/or additional laboratory data based at least in part on digital rock simulation results (e.g., fluid flow simulation results, etc.).

As an example, a digital rock model can be a model of rock as may occur naturally in the Earth or can be a model of material such as proppant. As an example, a model can be a hybrid model of rock and proppant. Types of proppant can include naturally occurring sand grains, man-made or specially engineered particles such as, for example, resin-coated sand or high-strength ceramic materials like sintered bauxite. Proppant materials can be sorted for size and sphericity to provide an efficient conduit for production of fluid from a reservoir to a wellbore.

As to chemicals that may be considered in a digital rock simulation of fluid flow and/or one or more other phenomena, a chemical can include one or more of the OpenFRAC fluid family of chemicals (Schlumberger Limited, Houston, Tex.). As an example, consider sodium chloride, magnesium chloride, amphoteric alkyl amine, calcium magnesium sodium phosphate, propan-2-ol, acrylamide copolymer, ammonium sulfate, sodium sulfate, potassium chloride, urea, hypochlorous acid, non-crystalline silica, dimethyl siloxanes, silicones, guar gum, hemicellulase (enzyme), boric acid, calcium chloride, etc.

As an example, a fluid can include one or more scale inhibitors that may act to reduce scaling of proppant. As an example, a fluid can provide for crosslinking, gel formation, linear gel formation, slickwater, etc. As an example, one or more chemicals can provide for drag reduction, load-water recovery, and/or formation stabilization. As an example, a chemical may provide for degradation of a component that is intended to be degraded during and/or after an operation.

As an example, a fluid may be formulated to facility transport of proppant (e.g., propping agent) in a fracture, may be formulated to be compatible with formation rock and fluid, may be formulated to generate enough pressure drop along a fracture to create a fracture of a desired width, may be formulated to minimize friction pressure losses during injection, may be formulated using chemical additives that are approved according to local environmental regulations, may be formulated to exhibit controlled-break to a low-viscosity fluid for cleanup after treatment, and may be formulated as to cost-effectiveness.

As an example, viscosity of a fluid may be optimized via chemical composition. As an example, density of a fluid may be optimized via chemical composition. As an example, viscosity and density of a fluid may be optimized via chemical composition. In such examples, optimization can include modeling of a proppant pack and simulating one or more physical phenomena, which can include flow, temperature, reaction rate or rates of various reactions, etc.

As an example, a method may optimize chemistry based at least in part on a type of fracture to be generated. For example, low-viscosity fluids pumped at high rates may aim to generate narrow, complex fractures with low-concentrations of propping agent (e.g., about 0.2 to about 5 lbm proppant added (PPA) per gallon (e.g., about 24 g/l to about 600 g/l)).

To minimize risk of premature screenout, a pumping rate can be selected to transport proppant over a desired distance, which may be along a horizontal wellbores. For a wide-biwing fracture, fluid can be selected to be of a viscosity for suspension and transport of higher proppant concentrations. Such a treatment fluid may be pumped at a lower pump rate and may create wider fractures (e.g., about 0.5 cm to about 2.5 cm).

Fluid density can affect the surface injection pressure and the ability of the fluid to flow back after treatment. In low-pressure reservoirs, low-density fluids, like foam, can be used to assist in fluid cleanup. Conversely, in certain deep reservoirs (including offshore), higher density fracturing fluids may be utilized.

Fracturing operations are one type of operations that can be planned, implemented, etc., based at least in part on results from a cloud-based digital rock simulation system. Other types of operations can include, for example, drilling operations, completions operations, data acquisition operations, etc.

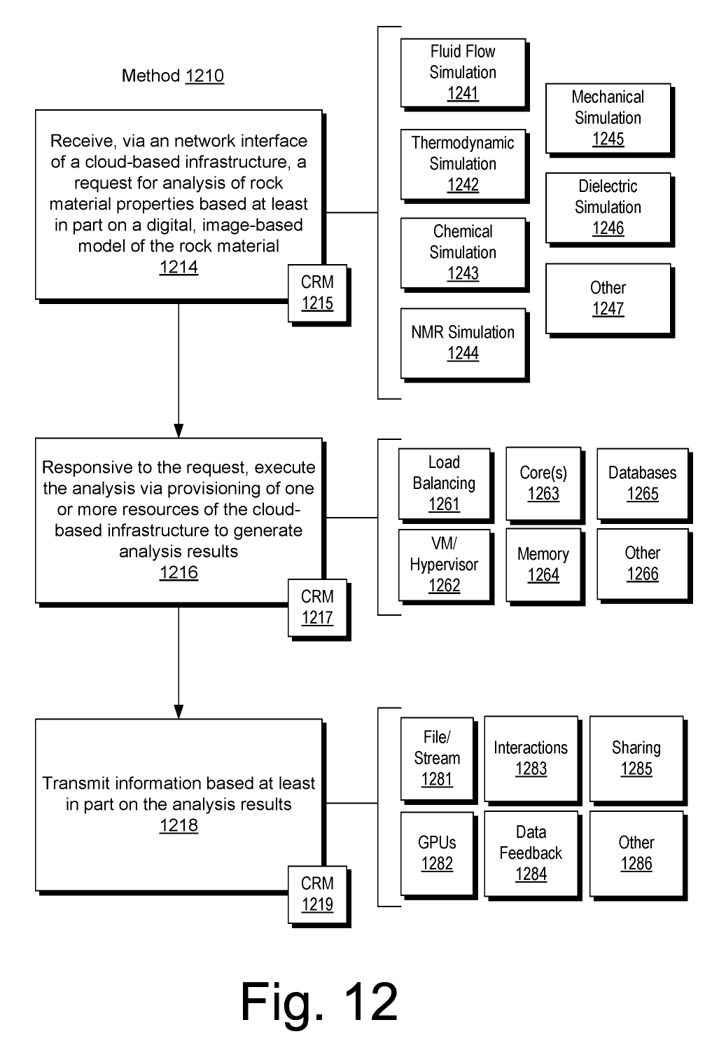

FIG. 12 shows an example of a method 1210 that includes a reception block 1214 for receiving, via an network interface of a cloud-based infrastructure, a request for analysis of rock material properties based at least in part on a digital, image-based model of the rock material; an execution block 1216 for, responsive to the request, executing the analysis via provisioning of one or more resources of the cloud-based infrastructure to generate analysis results; and a transmission block 1218 for transmitting information based at least in part on the analysis results.

As shown in FIG. 12, the reception block 1214 can receive a request that can specify a type of analysis or types of analyses. For example, consider one or more analyses associated with a fluid flow simulation block 1241 (e.g., DHD, etc.), a thermodynamic simulation block 1242, a chemical simulation block 1243 (e.g., of organic material, fluid chemicals, etc.), a nuclear magnetic resonance simulation block 1244 (e.g., proton NMR and/or one or more other types of NMR for one or more purposes such as, for example, diffusion, state(s) of water, phase(s), chemical reactions, etc.), a mechanical simulation block 1245 (e.g., geomechanical simulation, etc.), a dielectric simulation block 1246 (e.g., simulation of electrical properties, behaviors, etc.) and an other block 1247. As an example, a petrophysical simulation may include one or more types of simulations where, for example, various simulators are linked (e.g., instantiated via provisioned resources to generate petrophysical simulation results, etc.).

As shown in FIG. 12, the execution block 1216 can include various blocks for provisioning of resources and generation of analysis results. For example, consider a load balancing block 1261, a virtual machine and/or hypervisor block 1262, a core or cores provisioning block 1263, a memory provisioning block 1264, a databases provisioning block 1265 and an other block 1266. In such an example, the execution block 1216 can assess a request and provision resources to execute instructions to perform one or more analyses as associated with the request.

As shown in FIG. 12, the transmission block 1218 can include various blocks for transmission of analysis results. For example, consider a file and/or a stream block 1281 (e.g., to transmit a file, files, a data stream or data streams, as may be suitable for rendering using a media player, etc.), a GPUs block 1282 for utilizing one or more GPUs for generating renderable information (e.g., images and/or video for transmission), an interactions block 1283 for receiving information from a client device or client devices and adjusting transmission in response, a data feedback block 1284 for receiving data as feedback in response to analysis results (e.g., interpretation of analysis results, etc.), a sharing block 1285 for sharing information (e.g., joining one or more other client devices in a viewing/analysis session), and an other block 1286.

The method 1210 is shown in FIG. 12 in association with various computer-readable media (CRM) blocks 1215, 1217 and 1219. Such blocks generally include instructions suitable for execution by one or more processors (or cores) to instruct a computing device or system to perform one or more actions. While various blocks are shown, a single medium may be configured with instructions to allow for, at least in part, performance of various actions of the method 1210. As an example, a computer-readable medium (CRM) may be a computer-readable storage medium that is non-transitory and not a carrier wave.

As an example, a cloud-based infrastructure can operate according to one or more application programming interfaces (APIs). For example, a GUI rendered to a display of a client device can include graphical controls that are selectable to generate one or more API calls that can be received as a request or requests for analysis of rock material properties (see, e.g., the reception block 1214). As an example, such a GUI may include one or more viewing panes where information can be received as transmitted by a cloud-based infrastructure. In such an example, a viewing pane may render an image and/or render video (e.g., mpeg, avi or other format). As an example, interactions with the GUI may generate one or more API calls that can be received by the cloud-based infrastructure to cause the cloud-based infrastructure to adjust information, generate additional information (e.g., additional analysis results, additional images, additional video, etc.).

As an example, a method can include receiving, via an network interface of a cloud-based infrastructure, a request for analysis of rock material properties based at least in part on a digital, image-based model of the rock material; responsive to the request, executing the analysis via provisioning of one or more resources of the cloud-based infrastructure to generate analysis results; and transmitting information based at least in part on the analysis results. In such an example, the method can include building the digital, image-based model of the rock material based at least in part on a 3D digital image file of the rock material. For example, a cloud-based infrastructure can receive a 3D digital image file, a series of 2D digital image files, etc. as generated using one or more imaging modalities (e.g., X-ray, NMR, etc.) to image rock material (e.g., natural rock, proppant, etc.) and then building a digital, image-based model of rock material based on such image file or files (e.g., via segmentation, object recognition, positive and/or negative space recognition, etc.).

As an example, an analysis can be or include a direct hydrodynamic simulation of fluid flow in rock material. As an example, an analysis can be or include a simulation of geomechanical response of rock material to an applied load or applied loads (e.g., in one or more dimensions). As an example, an analysis can be or include a simulation of nuclear magnetic resonance of protons in the rock material. In such an example, the protons may be hydrocarbon protons and/or water protons. As an example, simulation can include flow simulation and/or diffusion simulation of fluid and/or particles that may include one or more types of protons. As an example, laboratory data and/or borehole data can include NMR data of fluid and/or solid/liquid suspensions in rock material. As an example, a simulation can be a simulation that generates synthetic NMR data that can be compared to actual NMR data. In such an example, a model of rock material may be refined, adjusted, etc. to cause a convergence between model-based results and NMR data where, for example, the model-based results may be based at least in part on a digital, image-based model of rock material.

As an example, an analysis can be an electrical analysis, a thermal conductivity analysis, a petrophysical process analysis or other type of analysis.

As an example, an analysis may analyze rock material with respect to one or more rock types that have been classified according to their petrophysical properties such as, for example, properties that pertain to fluid behavior within the rock, such as porosity, capillary pressure, permeabilities, irreducible saturations or saturations. As an example, petrophysical rock types may be analyzed with respect to core data and/or other data. As an example, data may include static data and/or dynamic data. As an example, a method can include executing an analysis based at least in part on one or more types of wireline log data. As an example, an analysis can include an electrofacies approach to determine one or more rock types.

As an example, an analysis can include interpreting petrophysical data. As an example, an analysis may generate analysis results as to one or more of shale volume, total porosity, effective porosity, water saturation and permeability.

As an example, rock material can include reservoir rock material, proppant material or reservoir rock material and proppant material.

As an example, a method can include accessing, via a cloud-based infrastructure, rock material data where the rock material data includes reservoir rock material data, proppant material data or reservoir rock material data and proppant material data. As an example, a method can include accessing, via a cloud-based infrastructure, fluid data and/or chemical data.

As an example, a method can include performing a sensitivity analysis for at least one reservoir property, at least one operational parameter or at least one reservoir property and at least one operational parameter. Such an analysis can be based at least in part on a digital, image-based model of rock material.

As an example, a method can include transmitting visualization information, which may be data, image data and/or video data.

As an example, an analysis can include a simulation of time-dependent behavior of fluid flow in rock material and/or a simulation of time-dependent mechanical behavior of rock material. In such a simulation or simulations, temperature, chemical concentration, etc., may vary with respect to time. As an example, an analysis can include a thermodynamic simulation.

As an example, a method can include receiving data where the data includes field data (e.g., borehole data), laboratory data or field data and laboratory data where, for example, an analysis can be based at least in part on a portion of the data. In such an example, the method can include transmitting a request for additional data based at least in part on the analysis results. Such a request may be, for example, to a client device in the field, a client device in a laboratory, etc. In response, the client device in the field and/or the client device in the laboratory may transmit the additional data to a cloud-based infrastructure.

As an example, a cloud-based infrastructure can include a network interconnect and servers operatively coupled to the network interconnect where the servers can include graphics processing units.

As an example, a system can include servers where each of the servers includes at least one processor, memory accessible by the at least one processor and processor-executable instructions stored in the memory to analyze rock material properties based on a digital, image-based model of the rock material to generate analysis results; a network interconnect wherein the servers are operatively coupled to the network interconnect; provisioning circuitry that provisions the servers responsive to receipt of a request to analyze the rock material properties; and transmission circuitry that transmits information based at least in part on the analysis results. In such an example, the servers can include graphics processing unit accelerators for three-dimensional data and the analysis results can include at least three-dimensional analysis results. As an example, the analysis results can include four-dimensional analysis results where the four dimensions include three spatial dimensions and time as a dimension. As an example, a cloud-based infrastructure may transmit four-dimensional information based at least in part on four-dimensional analysis results where such information may be transmitted as a file, files and/or as a stream or streams (e.g., streaming video, etc.).

As an example, one or more computer-readable storage media can include computer-executable instructions to instruct a computing system to: receive, via an network interface of a cloud-based infrastructure, a request for analysis of rock material properties based at least in part on a digital, image-based model of the rock material; responsive to the request, execute the analysis via provisioning of one or more resources of the cloud-based infrastructure to generate analysis results; and transmit information based at least in part on the analysis results.

Various types of equipment can include circuitry. The term “circuit” or “circuitry” can include all levels of available integration, e.g., from discrete logic circuits to the highest level of circuit integration such as VLSI, and includes programmable logic components programmed to perform the functions of an embodiment as well as general-purpose or special-purpose processors programmed with instructions to perform those functions. Circuitry can include one or more computer-readable media that include computer-executable instructions to instruct a computer to perform one or more actions. The term “computer-executable instructions” includes processor-executable instructions, whether a processor is a central processor, a graphics processor or other type of processor. Instructions stored on a computer-readable medium may be software (e.g., instructions for telling a computer, computing device, etc., what to do and how to do it). A computer-readable medium may be a storage device such as memory, an optical storage device, etc. Such a storage device may store instructions and optionally other information (e.g., data, etc.) in a non-transitory manner.

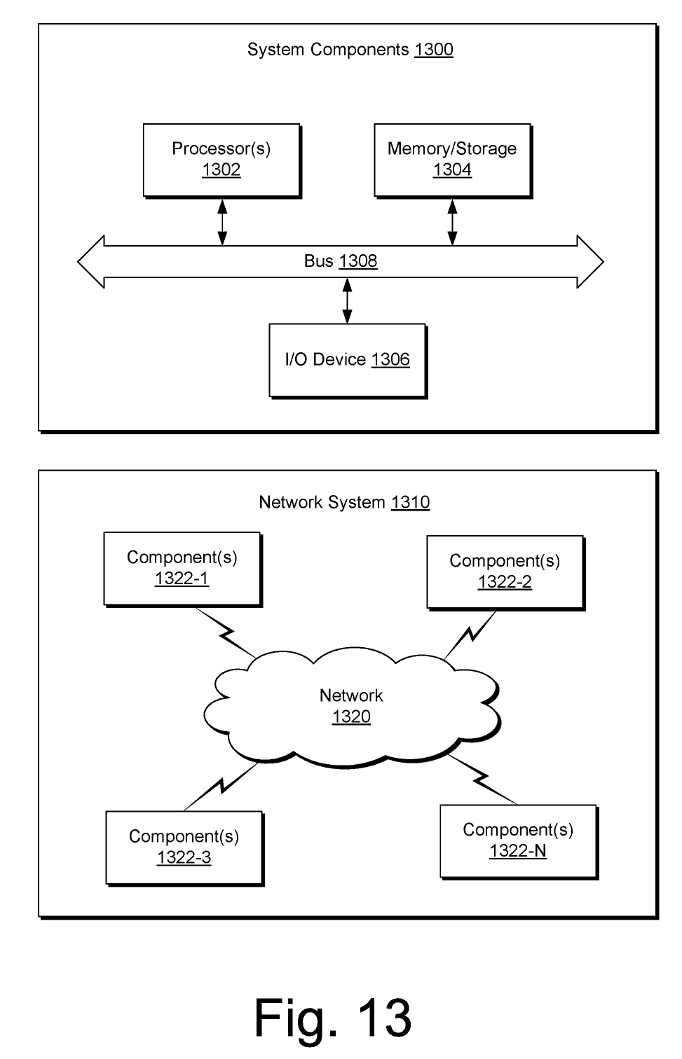

FIG. 13 shows components of an example of a computing system 1300 and an example of a networked system 1310. The system 1300 includes one or more processors 1302, memory and/or storage components 1304, one or more input and/or output devices 1306 and a bus 1308. In an example embodiment, instructions may be stored in one or more computer-readable media (e.g., memory/storage components 1304). Such instructions may be read by one or more processors (e.g., the processor(s) 1302) via a communication bus (e.g., the bus 1308), which may be wired or wireless. The one or more processors may execute such instructions to implement (wholly or in part) one or more attributes (e.g., as part of a method). A user may view output from and interact with a process via an I/O device (e.g., the device 1306). In an example embodiment, a computer-readable medium may be a storage component such as a physical memory storage device, for example, a chip, a chip on a package, a memory card, etc. (e.g., a computer-readable storage medium).

In an example embodiment, components may be distributed, such as in the network system 1310. The network system 1310 includes components 1322-1, 1322-2, 1322-3, . . . , 1322-N. For example, the components 1322-1 may include the processor(s) 1302 while the component(s) 1322-3 may include memory accessible by the processor(s) 1302. Further, the component(s) 1322-2 may include an I/O device for display and optionally interaction with a method. The network may be or include the Internet, an intranet, a cellular network, a satellite network, etc.

As an example, a device may be a mobile device that includes one or more network interfaces for communication of information. For example, a mobile device may include a wireless network interface (e.g., operable via IEEE 802.11, ETSI GSM, BLUETOOTH™, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, a system may be implemented on a distributed system having multiple nodes, where portions of the system may be located on different nodes within the distributed system. As an example, a node can correspond to a distinct computing device. As an example, a node can correspond to a computer processor with associated physical memory. As an example, a node may correspond to a computer processor or micro-core of a computer processor with shared memory and/or resources.

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that can be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few example embodiments have been described in detail above, those skilled in the art will readily appreciate that many modifications are possible in the example embodiments. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph 6 for any limitations of any of the claims herein, except for those in which the claim expressly uses the words “means for” together with an associated function.

What is claimed is:

1. A method comprising:
receiving, via an network interface of a cloud-based infrastructure, a request for analysis of rock material properties based at least in part on a digital, image-based model of the rock material;
responsive to the request, executing the analysis via provisioning of one or more resources of the cloud-based infrastructure to generate analysis results; and
transmitting information based at least in part on the analysis results.

2. The method of claim 1 comprising building the digital, image-based model of the rock material based at least in part on a 3D digital image file of the rock material.

3. The method of claim 1 wherein the analysis comprises a direct hydrodynamic simulation of fluid flow in the rock material.

4. The method of claim 1 wherein the analysis comprises a simulation of geomechanical response of the rock material to an applied load.

5. The method of claim 1 wherein the analysis comprises a simulation of nuclear magnetic resonance of protons in the rock material.

6. The method of claim 1 wherein the analysis comprises a member selected from a group consisting of electrical analysis, thermal conductivity analysis and petrophysical process analysis.

7. The method of claim 1 wherein the rock material comprises reservoir rock material, proppant material or reservoir rock material and proppant material.

8. The method of claim 1 comprising accessing, via the cloud-based infrastructure, rock material data wherein the rock material data comprises reservoir rock material data, proppant material data or reservoir rock material data and proppant material data.

9. The method of claim 1 comprising accessing, via the cloud-based infrastructure, fluid data.

10. The method of claim 1 comprising accessing, via the cloud-based infrastructure, chemical data.

11. The method of claim 1 comprising performing a sensitivity analysis for at least one reservoir property, at least one operational parameter or at least one reservoir property and at least one operational parameter.

12. The method of claim 1 wherein the transmitting information comprises transmitting visualization information.

13. The method of claim 1 wherein the analysis comprises a simulation of time-dependent behavior of fluid flow in the rock material.

14. The method of claim 1 receiving data wherein the data comprises field data, laboratory data or field data and laboratory data and wherein the analysis is based at least in part on a portion of the data.

15. The method of claim 14 comprising transmitting a request for additional data based at least in part on the analysis results.

16. The method of claim 1 wherein the cloud-based infrastructure comprises a network interconnect and servers operatively coupled to the network interconnect wherein the servers comprise graphics processing units.

17. The method of claim 1 wherein the analysis comprises a thermodynamic simulation.

18. A system comprising:
servers wherein each of the servers comprises at least one processor, memory accessible by the at least one processor and processor-executable instructions stored in the memory to analyze rock material properties based on a digital, image-based model of the rock material to generate analysis results;
a network interconnect wherein the servers are operatively coupled to the network interconnect;
provisioning circuitry that provisions the servers responsive to receipt of a request to analyze the rock material properties; and
transmission circuitry that transmits information based at least in part on the analysis results.

19. The system of claim 18 wherein the servers comprise graphics processing unit accelerators for three-dimensional data and wherein the analysis results comprise at least three-dimensional analysis results.

20. One or more computer-readable storage media comprising computer-executable instructions to instruct a computing system to:
receive, via an network interface of a cloud-based infrastructure, a request for analysis of rock material properties based at least in part on a digital, image-based model of the rock material;
responsive to the request, execute the analysis via provisioning of one or more resources of the cloud-based infrastructure to generate analysis results; and
transmit information based at least in part on the analysis results.
